(19) United States
(12) Patent Application Publication
(10) Pub. No.: US 2026/0185078 A1
(43) Pub. Date: Jul. 2, 2026

(54) LIBRARIES FOR RNA ENRICHMENT

(71) Applicant: Twist Bioscience Corporation, South San Francisco, CA (US)

(72) Inventors: Danny ANTAKI, South San Francisco, CA (US); Michael BOCEK, Inver Grove Heights, MN (US); Kristin D. BUTCHER, South San Francisco, CA (US); Yu CAI, South San Francisco, CA (US); Jean CHALLACOMBE, South San Francisco, CA (US); Derek MURPHY, South San Francisco, CA (US); Esteban TORO, Fremont, CA (US)

(73) Assignee: Twist Bioscience Corporation, South San Francisco, CA (US)

(21) Appl. No.: 19/116,608

(22) PCT Filed: Sep. 29, 2023
(86) PCT No.: PCT/US2023/075551

§ 371 Date: Mar. 28, 2025

Related U.S. Application Data
(60) Provisional application No. 63/377,667, filed on Sep. 29, 2022.
(60) Provisional application No. 63/482,230, filed on Jan. 30, 2023.

(51) Int. Cl. C12N 15/10

(52) U.S. Cl. CPC C12N 15/1051; C12N 15/1072

(57) ABSTRACT

Synthetic polynucleotide libraries may include a plurality of polynucleotides. The polynucleotides may comprise DNA and may be configured to hybridize with one or more regions of target nucleic acids. The target nucleic acids may comprise a cDNA library. The cDNA library may comprise at least one exon-exon boundary between a first exon and a second exon.

### CROSS-REFERENCE TO RELATED APPLICATIONS

This application is the national stage entry of International Patent Application No. PCT/US2023/075551, filed Sep. 29, 2023, which claims the benefits of priority to U.S. Provisional Patent Application No. 63/482,230, filed Jan. 30, 2023, and U.S. Provisional Patent Application No. 63/377,667, filed Sep. 29, 2022, the entirety of each of which are incorporated herein by reference. All publications, patents, and patent applications mentioned in this specification are herein incorporated by reference to the same extent as if each individual publication, patent, or patent application was specifically and individually indicated to be incorporated by reference.

### BACKGROUND

Sequencing of the transcriptome, or RNAseq, is an important and revolutionary tool to better understand the complexity of transcriptomics.

### SUMMARY

Provided herein are compositions and methods for analysis of RNA expression.

Provided herein are synthetic polynucleotide libraries comprising: a plurality of polynucleotides, wherein the polynucleotides comprise DNA and are configured to hybridize with one or more regions of target nucleic acids, and wherein the target nucleic acids comprise a cDNA library. Further provided herein are libraries wherein the cDNA library comprises at least one exon-exon boundary between a first exon and a second exon. Further provided herein are libraries wherein the plurality of polynucleotides comprises a first polynucleotide and a second polynucleotide, wherein the first and second polynucleotides do not span the at least one exon-exon boundary. Further provided herein are libraries wherein the first polynucleotide is configured to hybridize to the first exon, and the second polynucleotide is configured to hybridize to the second exon. Further provided herein are libraries wherein the plurality of polynucleotides comprises at least two polynucleotides which do not span at least 90% of exon-exon boundaries. Further provided herein are libraries wherein the plurality of polynucleotides comprises at least two polynucleotides which do not span any exon-exon boundaries. Further provided herein are libraries wherein the cDNA library is representative of at least 50,000 RNA transcripts. Further provided herein are libraries wherein the cDNA library is representative of 25,000 to 100,000 RNA transcripts. Further provided herein are libraries wherein the cDNA library is representative of at least 5,000 genes. Further provided herein are libraries wherein the cDNA library is representative of at least 10,000 genes. Further provided herein are libraries wherein the cDNA library is representative of 10,000 to 30,000 genes. Further provided herein are libraries wherein the polynucleotides are 80-160 bases in length. Further provided herein are libraries wherein the library comprises at least 50,000 polynucleotides. Further provided herein are libraries wherein the library comprises at least 500,000 polynucleotides. Further provided herein are libraries wherein the library comprises 100,000 to 750,000 polynucleotides. Further provided herein are libraries wherein the exon regions encode for at least 500 genes. Further provided herein are libraries wherein a portion of the genes comprise two or more isoforms. Further provided herein are libraries wherein the library further comprises the plurality of target nucleic acids. Further provided herein are libraries wherein at least a portion of the polynucleotides is biotinylated. Further provided herein are libraries wherein the library is configured to minimize hybridization with housekeeping genes. Further provided herein are libraries wherein housekeeping genes comprise the highest 1.5% expressed genes in a cell. Further provided herein are libraries wherein the target nucleic acids are derived from a human cell. Further provided herein are libraries wherein the target nucleic acids are derived from an FFPE sample. Further provided herein are libraries wherein the stoichiometry of the plurality of polynucleotides is adjusted based on mRNA transcript abundance. Further provided herein are libraries wherein the polynucleotides are tiled over the one or more exon regions. Further provided herein are libraries wherein library hybridization bias is minimized towards one or more exon-exon junctions.

Provided herein are methods for sequencing comprising: contacting a library provided herein with a sample comprising a plurality of target nucleic acids; enriching at least one nucleic acid that binds to the library; and sequencing the at least one enriched target nucleic acid. Further provided herein are methods wherein the method further comprises generating the target nucleic acids from RNA. Further provided herein are methods wherein the plurality of target nucleic acids comprise a cDNA library. Further provided herein are methods wherein the method does not comprise a ribosomal depletion step. Further provided herein are methods wherein sequencing results in no more than 10% intronic bases. Further provided herein are methods wherein sequencing results in no more than 2% rRNA bases. Further provided herein are methods wherein sequencing results in at least 80% expression profiling efficiency. Further provided herein are methods wherein sequencing results in no more than 10% duplication. Further provided herein are methods wherein sequencing results in no more than 1.5% incorrect read strands. Further provided herein are methods wherein sequencing results in no more than 3% median 3′ bias. Further provided herein are methods wherein at least 40% of sequenced bases are coding DNA sequences (CDS). Further provided herein are methods wherein at least 40% of sequenced bases are coding DNA sequences (CDS). Further provided herein are methods wherein the plurality of target nucleic acids is no more than 100 ng. Further provided herein are methods wherein the plurality of target nucleic acids is no more than 10 ng. Further provided herein are methods wherein sequencing comprises detection of at least one RNA fusion.

Provided herein are synthetic polynucleotide libraries comprising: a plurality of polynucleotides, wherein the polynucleotides comprise DNA and are configured to hybridize with one or more exon regions of target nucleic acids comprising RNA. Further provided herein are methods wherein the polynucleotides are 80-160 bases in length. Further provided herein are methods wherein the library comprises at least 50,000 polynucleotides. Further provided herein are methods wherein the library comprises 100,000 to 750,000 polynucleotides. Further provided herein are methods wherein the exon regions encode for at least 500 genes. Further provided herein are methods wherein a portion of the genes comprise two or more isoforms. Further provided herein are methods wherein the library further comprises the plurality of target nucleic acids. Further provided herein are methods wherein at least a portion of the polynucleotides is biotinylated. Further provided herein are methods wherein the library is configured to minimize hybridization with housekeeping genes. Further provided herein are methods wherein housekeeping genes comprise the highest 1.5% expressed genes in a cell. Further provided herein are methods wherein the cell is human. Further provided herein are methods wherein the stoichiometry of the plurality of polynucleotides is adjusted based on mRNA transcript abundance. Further provided herein are methods wherein the polynucleotides are tiled over the one or more exon regions. Further provided herein are methods wherein library hybridization bias is minimized towards one or more exon-exon junctions. Provided herein are method for sequencing comprising: contacting a library provided herein with a sample comprising a plurality of target nucleic acids, wherein the plurality of target nucleic acids comprises RNA; enriching at least one nucleic acid that binds to the library; and sequencing the at least one enriched target nucleic acid.

### BRIEF DESCRIPTION OF THE DRAWINGS

A better understanding of the features and advantages of the present subject matter will be obtained by reference to the following detailed description that sets forth illustrative embodiments and the accompanying drawings of which:

FIG. 1 shows a non-limiting example of a schematic in a design strategy comprising tiling according to some embodiments. A goal in the illustrated design strategy comprises avoiding bias in capturing different isoforms or novel fusions. In this design exons are longer than probe length and are tiled end-to-end. Exons are also between ½ probe length and full probe length and comprise print mismatches at ends. Exons are less than or equal to 40 nucleotides (nt) in length and can rely on shadow capture to cover.

FIG. 2 shows a non-limiting example of a schematic in a design strategy expression according to some embodiments. One opportunity to improve capture of low-expressed transcripts comprises removing (or reducing coverage of) housekeeping genes. Based on tissue-specific GTEx expression data in humans, taking out the top 1% of genes can make read depths 1.6-5 fold higher over the low-expressed transcripts.

FIG. 3 shows a non-limiting example of a schematic illustrating an RNA capture strategy according to come embodiments. The left diagram provides a hypothetical transcript containing 2 coding exons smaller than the probe size of 120 nt. Two probes can be placed at each end that terminate at either exon/exon boundary for short exons. The right diagram provides a schematic of probe tiling strategy against this region, where the long exons are tiled end-to-end.

FIGS. 4A and 4B show a non-limiting example of a schematic illustrating bias that can occur in a gene according to some embodiments. FIG. 4A illustrates a hypothetical gene from FIG. 3 with two probes including one directly targeting the known splice variant. FIG. 4B illustrates a fusion of that gene at the exon 1 junction with only one probe. The strategy can provide for at least one probe targeting fusions.

FIGS. 5A and 5B show a non-limiting example of a schematic illustrating an exon-aware tradeoff comprising isoform bias and expression bias according to some embodiments. Probes may not be evenly tiled across transcripts and may be placed with higher density near short exons. This can leads to significant discrepancies in probe density across the transcript, which may complicate expression analysis. FIG. 5A illustrates a density with low isoform bias and high expression bias, while FIG. 5B illustrates a density with high isoform bias and low expression bias.

FIG. 6 shows a non-limiting example of a sample correlation matrix according to some embodiments. Whole transcriptome sequencing (WTS) or exome captures did not correlate within a block, but WTS correlated generally well with the exome, and somewhat well between conditions. Exome captures correlated well with each other.

FIGS. 7A and 7B show a non-limiting example of expectations for capture (FIG. 7A) vs. the reality (FIG. 7B) according to some embodiments. It was expected that limited probe concentrations may produce a levelling effect at high expression (FIG. 7A), however capture roughly correlates with non-captured genes across many orders of magnitude (FIG. 7B). The overall improvement in capture was roughly 1.4-fold.

FIGS. 8A and 8B show a non-limiting example of uncaptured regions which are primarily non-targets according to some embodiments. The mean fragments per kilobase of transcript per million mapped reads (FPKM) are shown for capture vs no capture for as a function of exome coverage (FIG. 8A) and gene type (FIG. 8B). Most regions that are significantly lower in capture are non-target regions of Exome 2. Annotations are primarily long non-coding RNAs (lncRNAs).

FIGS. 9A and 9B show a non-limiting example of a splice variant bias according to some embodiments. There may be many examples of bias in capture. When only targeting coding sequence (CDS), differences in untranslated region (UTR) length can change outcomes.

FIG. 10 shows a non-limiting example of schematic illustrating a method for depletion using an RNA sequencing kit according to some embodiments. The method can comprise one or more steps including 1) Depletion: Homologous DNA sequences to rRNA+RNase H; 2) DNase I: DNase I; 3) RNA Fragmentation: Mg2++Heat; 4) First Strand Synthesis: M-MuLV or similar+Random Primers; 5) Second Strand Synthesis and A-Tailing: RNase H+DNA Polymerase I+DNA Ligase+T4 PNK+Taq DNA Polymerase; 6) Adapter Ligation: Universal Adapters with T overhangs+T4 DNA Ligase+PEG; and 7) Amplification using Barcoded Primers: High Fidelity Enzyme+Barcoded Primers. In some cases, about 100 ng of input universal human reference (UHR) RNA can be used.

FIG. 11 shows a non-limiting example of schematic illustrating a method for a RNA sequencing kit workflow according to some embodiments. The method can comprise one or more steps including 1) Fragmentation and First Strand Synthesis: Mg2++Heat+M-MuLV or similar+Random Primers; 2) Second Strand Synthesis and Adapter Ligation: 3′ Barcoded Primers homologous to Random Primers+High Fidelity Enzyme; 3) Depletion: Homologous DNA sequences to rRNA+dsDNA cleaving enzyme; and 4) Amplification using 5′ Barcoded Primers: High Fidelity Enzyme+5′ Barcoded Primers. In some cases, about 1 ng or about 10 ng of input UHR RNA can be used.

FIGS. 12A, 12B, and 12C show a non-limiting example of target enrichment (TE) with an RNA fusion panel according to some embodiments. Figures are provided for percent off pair (FIG. 12A), mean target coverage (FIG. 12B) and zero coverage targets percent (FIG. 12C). RNA libraries may be generated using different kits, including 1 ng, 10 ng, and 100 ng of input. PCR may be performed once or twice, where each PCR comprises about 5, 10, 13, or 15 cycles. The Takara SMART Seq included (1) 1 ng input-PCR1 at 5 cycles, PCR2 at 15 cycles; and (2) 10 ng input-PCR1 at 5 cycles, PCR2 at 13 cycles. The WM RNAseq Kit included 100 ng input-10 cycles. Duplicate captures were performed for each kit and input level using STv2. Sequencing was done on a Nextseq550 with 2×76 bp sequencing. WTS was also performed.

FIGS. 13A, 13B, 13C, and 13D show a non-limiting example of target enrichment (TE) with a higher burden of duplicate reads according to some embodiments. The RNAseq kit performed well with highest rates of uniquely mapped reads (FIG. 13A), PF bases (FIG. 13B), and low rate of chimeric reads (FIG. 13C). TE as a whole has a higher duplicate rate (FIG. 13D), which was in part driven by mass input.

FIGS. 14A, 14B, 14C, and 14D show a non-limiting example of target enrichment with a lower rate of rRNA reads according to some embodiments. The WM WTS has expected ˜5% rRNA abundance. It was expected to see lower rRNA rates for TE. Takara TE has a wide variation of reads unmapped too short, which are not necessarily contam. WM TE has slightly higher intergenic rate near target genes.

FIGS. 15A, 15B, and 15C show a non-limiting example of target enrichment where the WM TE sequences more UTR than Takara according to some embodiments. It was expected to see bad performance for WTS. Metrics were restricted to target genes. A higher intronic burden in WM can still be seen.

FIGS. 16A, 16B, and 16C show a non-limiting example of TE capturing more target gene sequences according to some embodiments. The number of reads detected for the 46 target genes are shown as dashed lines. The TE for both WM and Takara were similar with around 30× we start getting dropouts of genes.

FIG. 17 shows a non-limiting example of a heat map of TE capturing lowly expressed genes 1-2 orders of magnitude greater than WTS according to some embodiments. The characters in the cells are as follows: “°” gene has <100 reads; “<” gene has <10 reads; and “0” gene has 0 reads. The color of the cells are log 10 (TPM) values. The right hand side of the heatmap genes that are lowly expressed in WTS sample have gene expression in the TE samples.

FIGS. 18A and 18B show a non-limiting example of a heat map of TE having a higher duplicate read rate according to some embodiments. The percentage of reads aligned to the gene are plotted that are flagged as duplicates. Duplicate rate (FIG. 18B) was correlated to the input mass. WM TE at 100 ng has intermediate dup rate (30-50%), where Takara 10 ng is higher (70-85%), and Takara 1 ng being the highest (>90% duplicates).

FIG. 19 shows a non-limiting example of a figure depicting expression and read duplicate rates being correlated for higher mass TE according to some embodiments. WM TE (X) show increased duplicate rate for target genes with higher expression values. See similar trend with 10 ng Takara TE (circle). The 1 ng mass input did not appear to have this effect but the genes have very high duplicate rates. Duplicate rate did not appear to correlate with increased expression for WTS libraries.

FIG. 20 shows a non-limiting example of an experimental set of for library generation according to some embodiments. The library generation is provided for 80 bp vs. 120 bp testing.

FIGS. 21A and 21B show a non-limiting example of a library quality control (QC), showing the final concentrations (FIG. 21A) and fragment sizes (FIG. 21B), according to some embodiments.

FIGS. 22A, 22B, 22C, and 22D show a non-limiting example of capture and final quality control according to some embodiments. The results are shown for 10 ng replicates (FIGS. 22A and 22B) and 100 ng replicates (FIGS. 22C and 22D). The results are shown for 80 bp (FIGS. 22A and 22C)) and 120 bp (FIGS. 22B and 22D).

FIGS. 23A, 23B, 23C, and 23D show shows a non-limiting example of RNAseq metrics according to some embodiments. Generally similar performance is shown between 120 bp and 80 bp panels in terms of selecting bases from exons, which can be seen in expression_profiling_efficiency (FIG. 23B) and pct_usable_bases (FIG. 23D). There are some slight differences shown in total library complexity (80 bp is slightly lower). This may be in part due to a small increase in the total amount of reads mapping to ribosomal elements in the 80 bp panel compared to the 120 bp panel.

FIGS. 24A and 24B show a non-limiting example of an expression comparison according to some embodiments. The expression is shown as a heat map (FIG. 24A) and as well as scatter plots (FIG. 24B). The expression is shown for 10 ng vs 100 ng (FIG. 24A), as well as 80 bp vs 120 bp probes both with 100 ng input (FIG. 24B). The results indicate reproducibility of capture between different capture conditions. Generally similar trends to exome capture results are observed. High expression probes were selected using GTEx data, but did not seem to be the highest expression genes in this dataset.

FIGS. 25A and 25B show shows a non-limiting example of isoform quantification biases according to some embodiments. The results are shown for 10 ng (FIG. 25A) vs 100 ng (FIG. 25B) of input. Salmon was used to obtain isoform-specific expression counts. Using these results, genes were filtered with detectable differences in multiple targeted isoforms (21 genes total). Each transcript count was normalized out to the mean for the associated gene. Mean-squared error was calculated for the measurements in the 120 bp and 80 bp panels compared to uncaptured. Results did not appear to show a consistent benefit of 80 vs 120 bp, however, with a limited set of genes.

FIGS. 26A and 26B show a non-limiting example of capture results in the DNA space according to some embodiments. Capture was run both against transcript sequences (with exact probes) and hg38 (with estimated targets). The off-target shown is high for RNA-space alignment, which may be in part due to unincluded transcript variants (e.g., non-coding). The PCT OFF BAIT in DNA-space shown is similar for 80 vs 120 bp probes (FIG. 26A). FOLD-80 appears to be higher for the 80 bp probes (FIG. 26B).

FIG. 27 shows a non-limiting example of standard panel generation (top) vs. partial biotin panel generation (bottom) according to some embodiments. In some cases, the partial biotin panel generation can be utilized in order to minimize the overwhelming detection of housekeeping genes

FIGS. 28A and 28B show a non-limiting example of partial biotin panel generation according to some embodiments. A 120 bp housekeeping panel dilution plate is shown (FIG. 28A) with primers used (SEQ ID. NO. 1-4) (FIG. 28B). The partial biotin primer ratios tried include 1%, 5%, 10%, 20%, and 100%.

FIGS. 29A, 29B, 29C, and 29D show a non-limiting example of a partial biotin panel bioanalyzer QC according to some embodiments. The panels shown include 120 bp housekeeping panels with 1%, 5%, 10%, and 20% biotin (FIGS. 29A-D, respectively).

FIGS. 30A, 30B, and 30C show a non-limiting example of quibit results (FIG. 30A) and bioanalyzer results (FIGS. 30B and 30C) for a streptavidin bead clean up method according to some embodiments. The results are shown for a 120 bp panel with partial biotin primer ratios of 1%, 5%, 10%, 20%, and 100%.

FIGS. 31A and 31B show a non-limiting example of biotin/protein ratio using a biotin quantification kit according to some embodiments. The results are shown for a 120 bp panel with partial biotin primer ratios of 1%, 5%, 10%, 20%, and 100% (FIG. 31A). FIG. 31B shows results with a change in y-axis to show sensitivity, excluding the 120 bp panel with 100% biotin.

FIGS. 32A and 32B show a non-limiting example of partial biotin panel QC using streptavidin beads (FIG. 32A) vs from a biotin quantification kit (FIG. 32B) according to some embodiments. Both methods show noticeable differences between 100% biotinylated panels and partial biotin panels, which indicates both methods could be used for QC. Both streptavidin beads method and biotin quantification kit provide similar results/trend, suggesting similar performance. Outlier (10% biotin) in the streptavidin beads method may be in part due to factors including poor mixing before Qubit, uneven beads distribution, or Qubit HS kit sensitivity.

FIGS. 33A and 33B show a non-limiting example of partial biotin spike-in testing according to some embodiments. Results are shown for 10 ng RNA (FIG. 33A) and 100 ng RNA (FIG. 33B) as input. Libraries using 10 ng and 100 ng of input with UHR and ERCC. Tested was performed using STv2 Capture protocol with 4 μl of partially biotinylated panels at 0.2 fmol/reaction/probe as spike-in and 4 μl of subset panel, all at 120 bp length: 1%, 5%, 10%, 20%, and 100%.

FIGS. 34A, 34B, 34C, and 34D show a non-limiting example of overall metrics for testing with partial biotin according to some embodiments. Results are shown for pct_rna (FIG. 34A), uniquely_mapped_reads_pct (FIG. 34B), expression_profiling_efficiency (34C), and pct_usable_bases (FIG. 34D). Slightly more favorable metrics are seen in terms of usable bases for higher mass percent of biotin.

FIGS. 35A-35J show a non-limiting example of correlation between captured and uncaptured according to some embodiments. Results are shown for 100 ng input (FIGS. 35A-E) and 10 ng input (FIGS. 35F-35J). Results are shown for 1% (FIGS. 35A and 35F), 5% (FIGS. 35B and 35G), 10% (FIGS. 35C and 35H), 20% (FIGS. 35D and 35I), and 100% (FIGS. 35E and 35J), respectively.

FIGS. 36A, 36B, and 36C show a non-limiting example of partial biotin vs. standard subsets according to some embodiments. Comparison of the enrichment of non-biotin/biotin genes are shown.

The results show some agreement between the capture fraction and the input quantity of biotin. 5% biotin sample appeared to be slightly anomalously high, which may be due to processing. FIG. 36C provides qualitative metrics of the percent capture in biotin genes.

FIGS. 37A and 37B show a non-limiting example of percent of reads in captured vs non-captured (FIG. 37A) and the approximate read savings as the number of genes removed from a panel increases (FIG. 37B) according to some embodiments. Read savings from highly expressed genes show marginal improvements compared to savings from excluding intron-containing reads, and reads from non-coding transcripts. Relatively marginal benefit are obtained from trimming a large number of genes (about 2.7-fold with no partial biotin, about 3.1-fold with removal of top 300 protein-coding genes).

FIGS. 38A, 38B, and 38C show a non-limiting example of an exon aware NGS probe design for RNA capture according to some embodiments. Genomic DNA Target (FIG. 38A) can comprise short exons, which are single probe centered and comprise probe overhangs into introns (may not be ideal for RNA capture in some embodiments), as well as long exons which can comprise probes flush to exon boundary. The RNA transcript target (e.g., not exon aware) (FIG. 38B) can comprise an entire sequence that tiled end to end with probes, where probes can cross exon boundaries to differing degrees depending on transcript and isoform. This may not be ideal for novel RNA isoform or fusion detection, in some embodiments. The RNA transcript (e.g., exon aware) (FIG. 38C) comprises short exons with two probes that are flush with the start and end and extend into adjacent exon. All other probes are flush with exon boundaries. This can be ideal for RNA capture, novel isoform, and fusion detection, according to some embodiments.

FIG. 39A depicts a content curation process for the RNA exome.

FIG. 39B depicts a DNA-based tiling strategy, similar to what is adopted for most DNA-based exomes over two isoforms of an example gene.

FIG. 39C depicts a tiling of the transcript sequences with probes.

FIG. 39D depicts an exon-aware design strategy, which used for the RNA exome designs herein, over the two example transcripts.

FIG. 40A depicts a graph of a comparison of sequencing metrics for enriched, whole transcript, and 3′-counting methods on identical reference samples.

FIG. 40B depicts a graph of a breakdown of signal from 3′ counting, RNA exome, and WTS by genome compartment.

FIG. 40C depicts a graph of the correlation between RNA exome and WTS showing enrichment in raw counts per gene.

FIG. 41A depicts a graph of the exonic rate (expression profiling efficiency) from FFPE and UHR RNA at mass inputs of Ing, 10 ng and 100 ng. The error bars represent SEM (standard error of the mean) for FIGS. 41A-41D.

FIG. 41B depicts a graph of the percent duplication as determined from UMI and mapping position from FFPE and UHR RNA at mass inputs of Ing, 10 ng and 100 ng.

FIG. 41C depicts a graph of percent of reads mapping to the incorrect strand from FFPE and UHR RNA at mass inputs of Ing, 10 ng and 100 ng.

FIG. 41D depicts a graph of the number of detected protein-coding genes and defined by GenCode from FFPE and UHR RNA at mass inputs of Ing, 10 ng and 100 ng.

FIG. 42A depicts a summary of differential expression experiment design.

FIG. 42B depicts a correlation of tumor/normal fold-change estimated from WTS (x-axis) to tumor/normal fold-change estimated from RNA exome capture (y-axis).

FIG. 42C depicts a graph of the comparison of false discovery rate (FDR) adjusted p-values from differential expression experiment in WTS and RNA exome capture comparing significance in each experiment.

FIG. 42D depicts a graph of the number of genes with FDR-corrected p-value <0.01 in RNA exome and WTS experiments at both mass conditions (10 ng and 100 ng).

FIG. 43A depicts a genome browser view of reads aligned to an EML4-ALK fusion transcript present in a cell-line derived standard-dotted black line represents the gene breakpoint.

FIG. 43B depicts a genome browser view of reads aligned to an EML4-ALK fusion transcript present in a cell-line derived standard-dotted black line represents the gene breakpoint. An SLC43A-ROS1 fusion is also present in the cell line.

FIG. 43C depicts a graph of the ratio of fusion/normal transcripts from samples in both WTS and RNA-exome capture for EML4-ALK (left) and SLC43A-ROS1 (right).

### DETAILED DESCRIPTION

Provided herein are methods, systems, and compositions for libraries for RNA enrichment.

Sequencing of the transcriptome, or RNAseq, can provide an important and revolutionary tool to better understand the complexity of transcriptomics. For example, total RNA sequencing can provide a relatively unbiased view of the transcriptional state of a population of cells. However, most total RNA-seq experiments are contend with a large number of reads that are not helpful for gene-expression analysis, including reads from highly abundant non-coding transcripts (like the 7SK RNA, or ribosomal RNA), intronic reads from pre-mRNA, or contaminating genomic DNA. Target enrichment can provide a way to focus sequencing on the informative parts of the genome, allowing for a more sensitive detection of low-abundance transcripts and/or for profiling only specific genes of interest.

Provided herein are capture sequencing experiments using a RNA-specific exome panel, which uses a novel design strategy to target protein-coding isoforms in Gencode v41 Basic. In some instances, the novel design strategy allows targeting of all protein-coding isoforms in Gencode v41 Basic. In some instances, the design natively targets the transcriptome. In some instances, the design strategy also places probes to minimize bias towards known isoforms, and can allow for discovery of novel isoforms or fusion genes. In some instances, the design integrates hybrid capture technology to the workflow of RNAseq to decrease overall sequencing costs and increase sequencing final metrics. In some instances, the workflows provided herein can be used to evaluate transcriptome-wide panels, as well as smaller targeted panels. In some instances, libraries of polynucleotides are used to capture specific regions (e.g., CDS) of a cDNA library.

The panel performance can be evaluated through expression quantification. For example, expression quantification can show that relative transcript abundances are preserved after hybrid capture. In some instances, this can allow for accurate and reproducible quantification of transcripts that are present across many orders of magnitude. Additionally, the target approach can results in gains in sequencing efficiency, as well as can demonstrate the ability to capture novel structural variants, such as, for example, RNA fusions common in cancers. Additionally, bioinformatic approach can be used to evaluate capture performance in RNA space. In some instances, the bioinformatic approach comprises specific challenges in the analysis of RNA-seq experiments. In some instances, the RNA-based targeted enrichment provided herein provides an effective way to efficiently profile gene expression, detect gene fusions, or both.

A difference between RNA and DNA capture may include the nature of the target space. For example, since RNA is spliced, and different splice isoforms may be present in different samples, it may not straightforward to design probes that could potentially target a large family of isoforms for a given gene. Similarly, in some instances, poor probe design can prevent the discovery of unknown or novel isoforms, and also of fusion genes. In some instances, these isoforms or fusion genes can be therapeutic targets of interest in cancer.

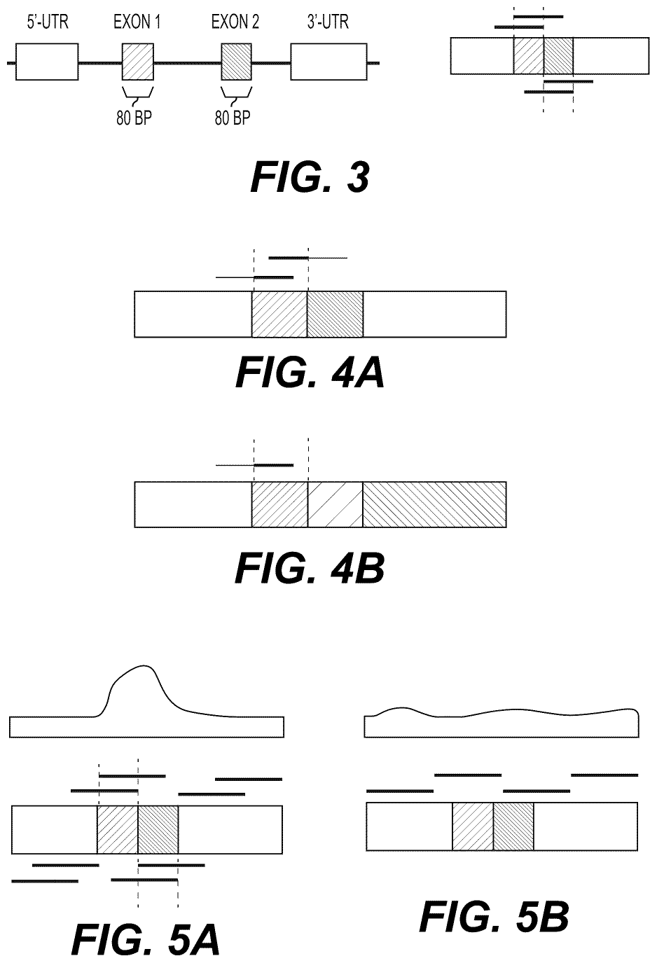

Provided herein is a strategy for placing probes across a transcript to minimize bias against novel splice junctions. An exemplary schematic of this design is shown in FIG. 3. For example, if an exon is larger than a probe size, the exon can be tiled end-to-end. In some instances, this is also done for DNA designs. As a further example, if the exon is smaller than the probe, two probes can be placed over the exon. In these examples, one may be designed flush against the left boundary of the exon, and the other may be designed flush against the right boundary of the exon. In this way, there can always be at least one probe for each splice junction that does not span the junction. In some instances, these probes can allow for a novel partner with this junction to be captured without significant bias.

Provided herein are processes for designing RNA capture panels. In some instances, the RNA capture panels can be used to understand the opportunities and limitations of RNA capture, as it relates to the uses of RNA-seq. In some instances, the RNA capture panels provide opportunities for use in single-cell RNA-seq (scRNA-seq). In some instances, the RNA capture panels provided herein may be used to detect rare SVs in low-expressed genes, rare isoforms of low-expressed genes, or both.

### Definitions

Throughout this disclosure, numerical features are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers+/−10% thereof, or 10% below the lower listed limit and 10% above the higher listed limit for the values listed for a range.

As used herein, the terms “preselected sequence”, “predefined sequence” or “predetermined sequence” are used interchangeably. The terms mean that the sequence of the polymer is known and chosen before synthesis or assembly of the polymer. In particular, various aspects of the invention are described herein primarily with regard to the preparation of nucleic acids molecules, the sequence of the oligonucleotide or polynucleotide being known and chosen before the synthesis or assembly of the nucleic acid molecules.

The term nucleic acid encompasses double- or triple-stranded nucleic acids, as well as single-stranded molecules. In double- or triple-stranded nucleic acids, the nucleic acid strands need not be coextensive (i.e., a double-stranded nucleic acid need not be double-stranded along the entire length of both strands). Nucleic acid sequences, when provided, are listed in the 5′ to 3′ direction, unless stated otherwise. Methods described herein provide for the generation of isolated nucleic acids. Methods described herein additionally provide for the generation of isolated and purified nucleic acids. The length of polynucleotides, when provided, are described as the number of bases and abbreviated, such as nt (nucleotides), bp (bases), kb (kilobases), or Gb (gigabases).

Provided herein are methods and compositions for production of synthetic (i.e. de novo synthesized or chemically synthesizes) polynucleotides. The term oligonucleic acid, oligonucleotide, oligo, and polynucleotide are defined to be synonymous throughout. Libraries of synthesized polynucleotides described herein may comprise a plurality of polynucleotides collectively encoding for one or more genes or gene fragments. In some instances, the polynucleotide library comprises coding or non-coding sequences. In some instances, the polynucleotide library encodes for a plurality of cDNA sequences. Reference gene sequences from which the cDNA sequences are based may contain introns, whereas cDNA sequences exclude introns. Polynucleotides described herein may encode for genes or gene fragments from an organism. Exemplary organisms include, without limitation, prokaryotes (e.g., bacteria) and eukaryotes (e.g., mice, rabbits, humans, and non-human primates). In some instances, the polynucleotide library comprises one or more polynucleotides, each of the one or more polynucleotides encoding sequences for multiple exons. Each polynucleotide within a library described herein may encode a different sequence, i.e., non-identical sequence. In some instances, each polynucleotide within a library described herein comprises at least one portion that is complementary to sequence of another polynucleotide within the library. Polynucleotide sequences described herein may be, unless stated otherwise, comprise DNA or RNA. A polynucleotide library described herein may comprise at least 10, 20, 50, 100, 200, 500, 1,000, 2,000, 5,000, 10,000, 20,000, 30,000, 50,000, 100,000, 200,000, 500,000, 1,000,000, or more than 1,000,000 polynucleotides. A polynucleotide library described herein may have no more than 10, 20, 50, 100, 200, 500, 1,000, 2,000, 5,000, 10,000, 20,000, 30,000, 50,000, 100,000, 200,000, 500,000, or no more than 1,000,000 polynucleotides. A polynucleotide library described herein may comprise 10 to 500, 20 to 1000, 50 to 2000, 100 to 5000, 500 to 10,000, 1,000 to 5,000, 10,000 to 50,000, 100,000 to 500,000, or to 50,000 to 1,000,000 polynucleotides. A polynucleotide library described herein may comprise about 370,000; 400,000; 500,000 or more different polynucleotides.

Provided herein are methods and compositions for production of synthetic (i.e. de novo synthesized) genes. Libraries comprising synthetic genes may be constructed by a variety of methods described in further detail elsewhere herein, such as PCA, non-PCA gene assembly methods or hierarchical gene assembly, combining (“stitching”) two or more double-stranded polynucleotides to produce larger DNA units (i.e., a chassis). Libraries of large constructs may involve polynucleotides that are at least 1, 1.5, 2, 3, 4, 5, 6, 7, 8, 9, 10, 15, 20, 30, 40, 50, 60, 70, 80, 90, 100, 125, 150, 175, 200, 250, 300, 400, 500 kb long or longer. The large constructs can be bounded by an independently selected upper limit of about 5000, 10000, 20000 or 50000 base pairs. The synthesis of any number of polypeptide-segment encoding nucleotide sequences, including sequences encoding non-ribosomal peptides (NRPs), sequences encoding non-ribosomal peptide-synthetase (NRPS) modules and synthetic variants, polypeptide segments of other modular proteins, such as antibodies, polypeptide segments from other protein families, including non-coding DNA or RNA, such as regulatory sequences e.g. promoters, transcription factors, enhancers, siRNA, shRNA, RNAi, miRNA, small nucleolar RNA derived from microRNA, or any functional or structural DNA or RNA unit of interest. The following are non-limiting examples of polynucleotides: coding or non-coding regions of a gene or gene fragment, intergenic DNA, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), small nucleolar RNA, ribozymes, complementary DNA (cDNA), which is a DNA representation of mRNA, usually obtained by reverse transcription of messenger RNA (mRNA) or by amplification; DNA molecules produced synthetically or by amplification, genomic DNA, recombinant polynucleotides, branched polynucleotides, plasmids, vectors, isolated DNA of any sequence, isolated RNA of any sequence, nucleic acid probes, and primers. cDNA encoding for a gene or gene fragment referred to herein, may comprise at least one region encoding for exon sequence(s) without an intervening intron sequence found in the corresponding genomic sequence. Alternatively, the corresponding genomic sequence to a cDNA may lack an intron sequence in the first place.

### Polynucleotide Probe Structures

Libraries of polynucleotide probes can be used to enrich particular target sequences in a larger population of sample polynucleotides. In some instances, polynucleotide probes each comprise an target binding sequence complementary to one or more target sequences, one or more non-target binding sequences, and one or more primer binding sites, such as universal primer binding sites. Target binding sequences that are complementary or at least partially complementary in some instances bind (hybridize) to target sequences.

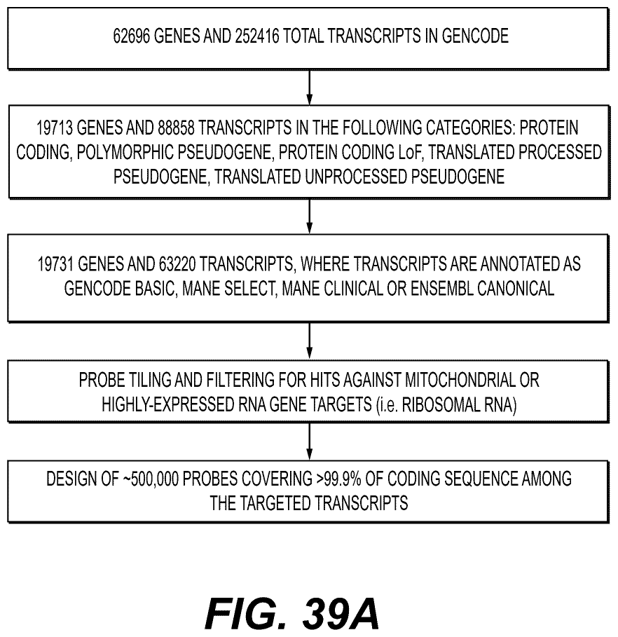

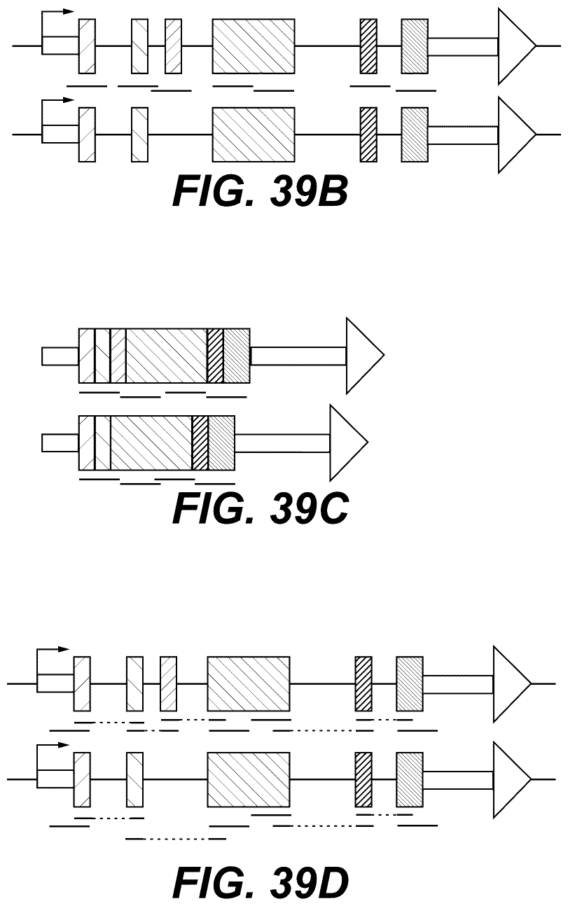

Provided herein are synthetic polynucleotide libraries comprising a plurality of polynucleotides. In some instances, the polynucleotides comprise DNA. In some instances, the polynucleotides are configured to hybridize with one or more regions of target nucleic acids. In some instances, target nucleic acids comprise a cDNA library. In some instances, probe designs are shown in FIG. 39B-39D. In some instances the cDNA library comprises at least one exon-exon boundary between a first exon and a second exon. In some instances, the synthetic polynucleotide library comprises at least two polynucleotides which do not span the at least one exon-exon boundary. In some instances, at least one polynucleotide is configured to hybridize to the first exon, and at least one polynucleotide is configured to hybridize to the second exon. In some instances, at least two polynucleotides which do not span at least 90% of exon-exon boundaries. In some instances, at least two polynucleotides which do not span any exon-exon boundaries. In some instances, the plurality of polynucleotides is adjusted based on mRNA transcript abundance. In some instances, polynucleotides are tiled over the one or more exon regions. In some instances, library hybridization bias is minimized towards one or more exon-exon junctions.

cDNA libraries may comprise a plurality of transcripts which can be targeted by polynucleotide probe libraries described herein. In some instances, the cDNA library is representative of at least 5,000, 10,000, 20,000, 25,000, 30,000, 35,000, 40,000, 45,000, 50,000, 55,000, 60,000, 70,000, 80,000, 90,000, or at least 100,000 RNA transcripts. In some instances the cDNA library is representative of 25,000 to 50,000, 25,000 to 75,000, 25,000 to 100,000, 5,000 to 75,000, 5,000 to 50,000, 10,000 to 50,000, 10,000 to 30,000, or 10,000 to 75,000 RNA transcripts. A cDNA libraries in some instances is representative of at least 500, 750, 1000, 1500, 2000, 2500, 3000, 3500, 4000, 5,000, 5500, 6000, 7000, 8000, 9000, or at least 10,000 genes. A cDNA libraries in some instances is representative of 5,000 to 10,000, 5,000 to 15,000, 5,000 to 20,000, 5,000 to 30,000, 10,000 to 30,000, or 10,000 to 40,000 genes. In some instances, a portion of the genes comprise two or more isoforms.

Polynucleotide probes may be configured to bind to regions of cDNA. In some instances, regions comprise CDS (coding DNA sequences). In some instances, probes are configured to minimize hybridization with housekeeping genes. In some instances, housekeeping genes comprise the highest 0.1%, 0.2%, 0.3%, 0.5%, 1%, 1.2%, 1.5%, 1.75%, 2%, or 2.5% expressed genes in a cell.

cDNA (target nucleic acids) may be derived from any sample source described herein. In some instances, the cDNA is derived from a cell. In some instances, the cell comprises a human cell. In some instances cDNA is derived from a formalin-fixed paraffin-embedded (FFPE) sample. In some instances, the polynucleotide probes provided herein can recover coding sequences from a sample comprising damaged nucleic acids (e.g., FFPE sample).

In some instances, the polynucleotide probes provided herein can reduce duplicate rates, reduce incorrect strand percent, or increase the number of detected genes compared to whole transcriptome sequencing (WTC). In some instances, the polynucleotides provided herein detect novel fusions.

Primer binding sites, such as universal primer binding sites facilitate simultaneous amplification of all members of the probe library, or a subpopulation of members. In some instances, the probes further comprise a barcode or index sequence. Barcodes are nucleic acid sequences that allow some feature of a polynucleotide with which the barcode is associated to be identified. After sequencing, the barcode region provides an indicator for identifying a characteristic associated with the coding region or sample source. Barcodes can be designed at suitable lengths to allow sufficient degree of identification, e.g., at least about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, or more bases in length. Multiple barcodes, such as about 2, 3, 4, 5, 6, 7, 8, 9, 10, or more barcodes, may be used on the same molecule, optionally separated by non-barcode sequences. In some embodiments, each barcode in a plurality of barcodes differ from every other barcode in the plurality at least three base positions, such as at least about 3, 4, 5, 6, 7, 8, 9, 10, or more positions. In some instances, the polynucleotides are ligated to one or more molecular (or affinity) tags such as a small molecule, peptide, antigen, metal, or protein to form a probe for subsequent capture of the target sequences of interest. In some instances, two probes that possess complementary target binding sequences which are capable of hybridization form a double stranded probe pair.

Probes described here may be complementary to target sequences which are sequences in a genome. Probes described here may be complementary to target sequences which are exome sequences in a genome. Probes described here may be complementary to target sequences which are intron sequences in a genome. In some instances, probes comprise an target binding sequence complementary to a target sequence, and at least one non-target binding sequence that is not complementary to the target. In some instances, the target binding sequence of the probe is about 120 nucleotides in length, or at least 10, 15, 20, 25, 50, 75, 100, 110, 120, 125, 140, 150, 160, 175, 200, 300, 400, 500, or more than 500 nucleotides in length. The target binding sequence is in some instances no more than 10, 15, 20, 25, 50, 75, 100, 125, 150, 175, 200, or no more than 500 nucleotides in length. The target binding sequence of the probe is in some instances about 120 nucleotides in length, or about 10, 15, 20, 25, 40, 50, 60, 70, 80, 85, 87, 90, 95, 97, 100, 105, 110, 115, 117, 118, 119, 120, 121, 122, 123, 124, 125, 126, 127, 128, 129, 130, 135, 140, 145, 150, 155, 157, 158, 159, 160, 161, 162, 163, 164, 165, 166, 167, 168, 169, 170, 175, 180, 190, 200, 210, 220, 230, 240, 250, 300, 400, or about 500 nucleotides in length. The target binding sequence is in some instances about 20 to about 400 nucleotides in length, or about 30 to about 175, about 40 to about 160, about 50 to about 150, about 75 to about 130, about 90 to about 120, or about 100 to about 140 nucleotides in length. The non-target binding sequence(s) of the probe is in some instances at least about 20 nucleotides in length, or at least about 1, 5, 10, 15, 17, 20, 23, 25, 50, 75, 100, 110, 120, 125, 140, 150, 160, 175, or more than about 175 nucleotides in length. The non-target binding sequence often is no more than about 5, 10, 15, 20, 25, 50, 75, 100, 125, 150, 175, or no more than about 200 nucleotides in length. The non-target binding sequence of the probe often is about 20 nucleotides in length, or about 1, 2, 3, 4, 5, 6, 7, 8, 9, 10, 11, 12, 13, 14, 15, 16, 17, 18, 19, 20, 21, 22, 23, 25, 40, 50, 60, 70, 80, 90, 100, 110, 120, 130, 140, 150, or about 200 nucleotides in length. The non-target binding sequence in some instances is about 1 to about 250 nucleotides in length, or about 20 to about 200, about 10 to about 100, about 10 to about 50, about 30 to about 100, about 5 to about 40, or about 15 to about 35 nucleotides in length. The non-target binding sequence often comprises sequences that are not complementary to the target sequence, and/or comprise sequences that are not used to bind primers. In some instances, the non-target binding sequence comprises a repeat of a single nucleotide, for example polyadenine or polythymidine. A probe often comprises none or at least one non-target binding sequence. In some instances, a probe comprises one or two non-target binding sequences. The non-target binding sequence may be adjacent to one or more target binding sequences in a probe. For example, an non-target binding sequence is located on the 5′ or 3′ end of the probe. In some instances, the non-target binding sequence is attached to a molecular tag or spacer.

In some instances, the non-target binding sequence(s) may be a primer binding site. The primer binding sites often are each at least about 20 nucleotides in length, or at least about 10, 12, 14, 16, 18, 20, 22, 24, 26, 28, 30, 32, 34, 36, 38, or at least about 40 nucleotides in length. Each primer binding site in some instances is no more than about 10, 12, 14, 16, 18, 20, 22, 24, 26, 28, 30, 32, 34, 36, 38, or no more than about 40 nucleotides in length. Each primer binding site in some instances is about 10 to about 50 nucleotides in length, or about 15 to about 40, about 20 to about 30, about 10 to about 40, about 10 to about 30, about 30 to about 50, or about 20 to about 60 nucleotides in length. In some instances the polynucleotide probes comprise at least two primer binding sites. In some instances, primer binding sites may be universal primer binding sites, wherein all probes comprise identical primer binding sequences at these sites. In some instances, a pair of polynucleotide probes targeting a particular sequence and its reverse complement (e.g., a region of genomic DNA) comprise a first target binding sequence, a second target binding sequence, a first non-target binding sequence, and a second non-target binding sequence. For example, a pair of polynucleotide probes complementary to a particular sequence (e.g., a region of genomic DNA).

In some instances, the first target binding sequences the reverse complement of the second target binding sequence. In some instances, both target binding sequences are chemically synthesized prior to amplification. In an alternative arrangement, a pair of polynucleotide probes targeting a particular sequence and its reverse complement (e.g., a region of genomic DNA) comprise a first target binding sequence, a second target binding sequence, a first non-target binding sequence, a second non-target binding sequence, a third non-target binding sequence, and a fourth non-target binding sequence. In some instances, the first target binding sequence is the reverse complement of the second target binding sequence. In some instances, one or more non-target binding sequences comprise polyadenine or polythymidine.

In some instances, both probes in the pair are labeled with at least one molecular tag. In some instances, PCR is used to introduce molecular tags (via primers comprising the molecular tag) onto the probes during amplification. In some instances, the molecular tag comprises one or more biotin, folate, a polyhistidine, a FLAG tag, glutathione, or other molecular tag consistent with the specification. In some instances probes are labeled at the 5′ terminus. In some instances, the probes are labeled at the 3′ terminus. In some instances, both the 5′ and 3′ termini are labeled with a molecular tag. In some instances, the 5′ terminus of a first probe in a pair is labeled with at least one molecular tag, and the 3′ terminus of a second probe in the pair is labeled with at least one molecular tag. In some instances, a spacer is present between one or more molecular tags and the nucleic acids of the probe. In some instances, the spacer may comprise an alkyl, polyol, or polyamino chain, a peptide, or a polynucleotide. The solid support used to capture probe-target nucleic acid complexes in some instances, is a bead or a surface. The solid support in some instances comprises glass, plastic, or other material capable of comprising a capture moiety that will bind the molecular tag. In some instances, a bead is a magnetic bead. For example, probes labeled with biotin are captured with a magnetic bead comprising streptavidin. The probes are contacted with a library of nucleic acids to allow binding of the probes to target sequences. In some instances, blocking polynucleic acids are added to prevent binding of the probes to one or more adapter sequences attached to the target nucleic acids. In some instances, blocking polynucleic acids comprise one or more nucleic acid analogues. In some instances, blocking polynucleic acids have a uracil substituted for thymine at one or more positions.

Probes described herein may comprise complementary target binding sequences which bind to one or more target nucleic acid sequences. In some instances, the target sequences are any DNA or RNA nucleic acid sequence. In some instances, target sequences may be longer than the probe insert. In some instance, target sequences may be shorter than the probe insert. In some instance, target sequences may be the same length as the probe insert. For example, the length of the target sequence may be at least or about at least 2, 10, 15, 20, 25, 30, 35, 40, 45, 50, 100, 150, 200, 300, 400, 500, 1000, 2000, 5,000, 12,000, 20,000 nucleotides, or more. The length of the target sequence may be at most or about at most 20,000, 12,000, 5,000, 2,000, 1,000, 500, 400, 300, 200, 150, 100, 50, 45, 35, 30, 25, 20, 19, 18, 17, 16, 15, 14, 13, 12, 11, 10, 2 nucleotides, or less. The length of the target sequence may fall from 2-20,000, 3-12,000, 5-5, 5000, 10-2,000, 10-1,000, 10-500, 9-400, 11-300, 12-200, 13-150, 14-100, 15-50, 16-45, 17-40, 18-35, and 19-25. The probe sequences may target sequences associated with specific genes, diseases, regulatory pathways, or other biological functions consistent with the specification.

In some instances, a single probe insert is complementary to one or more target sequences in a larger polynucleic acid. An exemplary target sequence is an exon. In some instances, one or more probes target a single target sequence. In some instances, a single probe may target more than one target sequence. In some instances, the target binding sequence of the probe targets both a target sequence and an adjacent sequence. In some instances, a first probe targets a first region and a second region of a target sequence, and a second probe targets the second region and a third region of the target sequence. In some instances, a plurality of probes targets a single target sequence, wherein the target binding sequences of the plurality of probes contain one or more sequences which overlap with regard to complementarity to a region of the target sequence. In some instances, probe inserts do not overlap with regard to complementarity to a region of the target sequence. In some instances, at least at least 2, 10, 15, 20, 25, 30, 35, 40, 45, 50, 100, 150, 200, 300, 400, 500, 1000, 2000, 5,000, 12,000, 20,000, or more than 20,000 probes target a single target sequence. In some instances no more than 4 probes directed to a single target sequence overlap, or no more than 3, 2, 1, or no probes targeting a single target sequence overlap. In some instances, one or more probes do not target all bases in an target sequence, leaving one or more gaps. In some instances, the gaps are near the middle of the target sequence. In some instances, the gaps are at the 5′ or 3′ ends of the target sequence. In some instances, the gaps are 6 nucleotides in length. In some instances, the gaps are no more than 1, 2, 3, 4, 5, 6, 7, 8, 9, 10, 20, 30, 40, or no more than 50 nucleotides in length. In some instances, the gaps are at least 1, 2, 3, 4, 5, 6, 7, 8, 9, 10, 20, 30, 40, or at least 50 nucleotides in length. In some instances, the gaps length falls within 1-50, 1-40, 1-30, 1-20, 1-10, 2-30, 2-20, 2-10, 3-50, 3-25, 3-10, or 3-8 nucleotides in length. In some instances, a set of probes targeting a sequence do not comprise overlapping regions amongst probes in the set when hybridized to complementary sequence. In some instances, a set of probes targeting a sequence do not have any gaps amongst probes in the set when hybridized to complementary sequence. Probes may be designed to maximize uniform binding to target sequences. In some instances, probes are designed to minimize target binding sequences of high or low GC content, secondary structure, repetitive/palindromic sequences, or other sequence feature that may interfere with probe binding to a target. In some instances, a single probe may target a plurality of target sequences.

A probe library described herein may comprise at least 10, 20, 50, 100, 200, 500, 1,000, 2,000, 5,000, 10,000, 20,000, 50,000, 100,000, 200,000, 500,000, 1,000,000 or more than 1,000,000 probes. A probe library may have no more than 10, 20, 50, 100, 200, 500, 1,000, 2,000, 5,000, 10,000, 20,000, 50,000, 100,000, 200,000, 500,000, or no more than 1,000,000 probes. A probe library may comprise 10 to 500, 20 to 1000, 50 to 2000, 100 to 5000, 500 to 10,000, 1,000 to 5,000, 10,000 to 50,000, 100,000 to 500,000, or to 50,000 to 1,000,000 probes. A probe library may comprise about 370,000; 400,000; 500,000 or more different probes.

### Next Generation Sequencing Applications

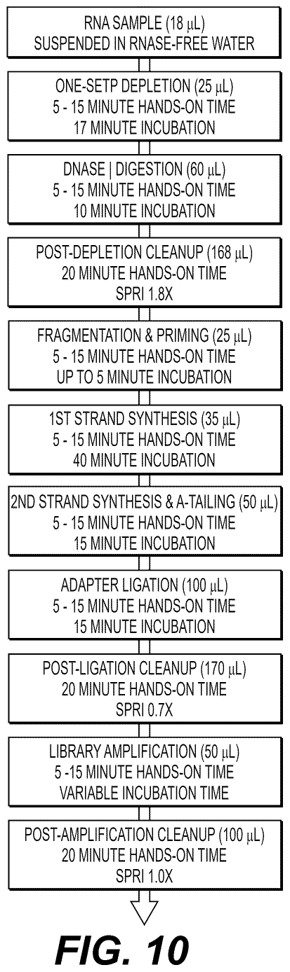

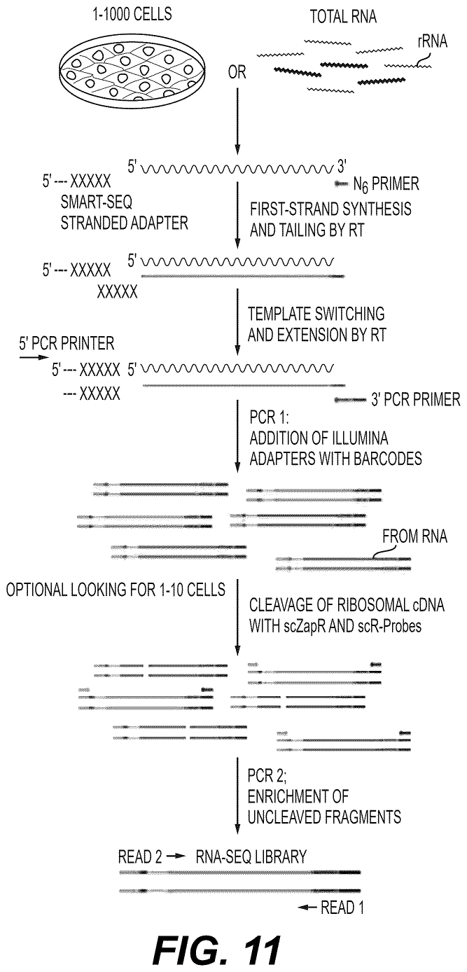

Provided herein are methods for enrichment and sequencing of nucleic acids. In some instances, nucleic acids comprise a cDNA library derived from RNA. In some instances, an exemplary workflow for cDNA library preparation is shown in FIG. 10 or FIG. 11. In some instances, preparation of a cDNA library comprises one or more steps of obtaining an RNA sample, depleting ribosomal RNA (rRNA), DNA digestion (e.g., DNase I), post-depletion cleanup, fragmentation and priming, first strand synthesis, 2nd strand synthesis, A-tailing, adapter ligation, post-ligation cleanup, cDNA library amplification, post-amplification cleaning. In some instances a cDNA library is then contacted with a polynucleotide (probe) library described herein to enrich target nucleic acids. In some instances cDNA library preparation comprises an RNASeq workflow.

Provided herein are methods for sequencing comprising one or more steps of contacting a library provided herein with a sample comprising a plurality of target nucleic acids; enriching at least one nucleic acid that binds to the library; and sequencing the at least one enriched target nucleic acid. In some instances, the target nucleic acids are generated or derived from RNA. In some instances the plurality of target nucleic acids comprise a cDNA library. Enrichment with a library provided herein in some instances reduces the amount of rRNA in a cDNA library. In some instances the method does not comprise a ribosomal depletion step. In some instances the method does not comprise a ribosomal depletion step in addition to enrichment. In some instances rRNA depletion comprises enrichment based on poly(T) or removal of rRNA. In some instances, removal of rRNA comprises binding probes to rRNA to separate the rRNA from the remainder of the RNA.

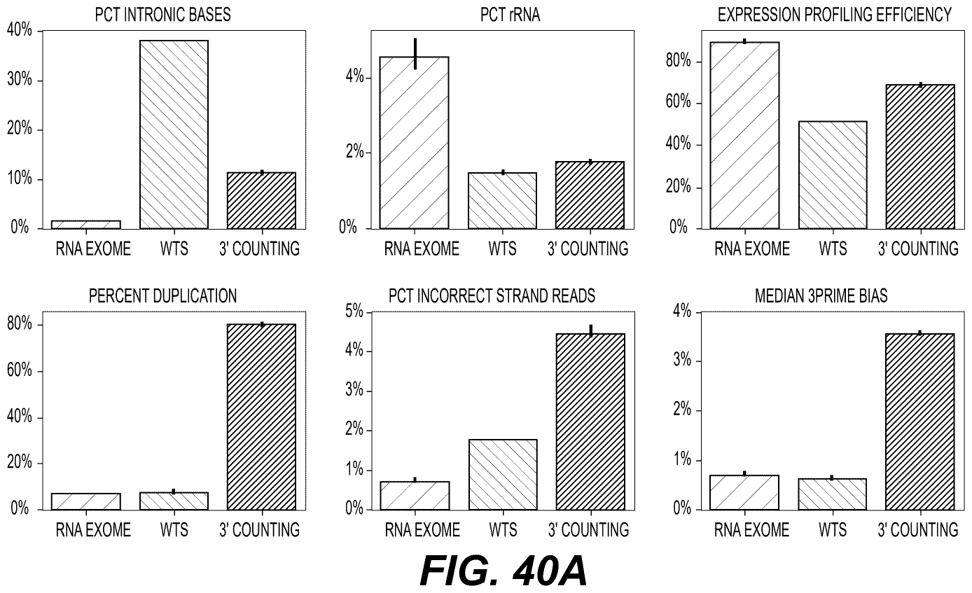

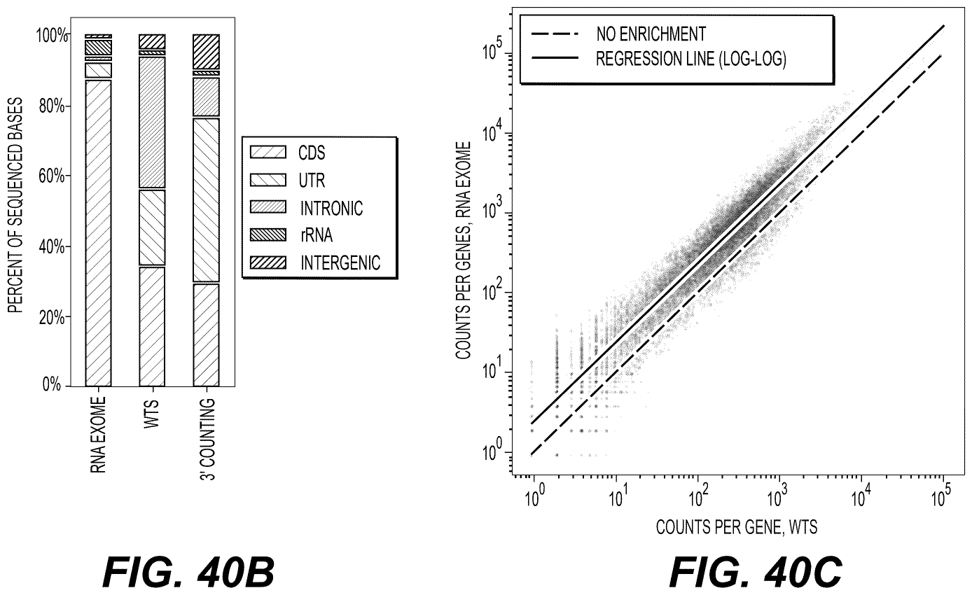

Use of a polynucleotide library provided herein may result in improved sequencing outcomes. In some instances, outcomes are improved relative to WTS or 3′ counting methods (FIGS. 40A-40C). In some instances sequencing results in no more than 1%, 2%, 3%, 4%, 5%, 7%, 10%, 12%, 15%, or no more than 20% intronic bases. In some instances sequencing results in 1-20%, 1-15%, 1-12%, 1-10%, 1-8%, 1-7%, 1-5%, 1-3%, 2-5%, 2-10%, 0.5-10%, 0.5-5%, 0.5-3%, 0.1-3%, 0.1-2%, or 0.1-1.5% intronic bases. In some instances sequencing results in no more than 15%, 10%, 8%, 7%, 6%, 5%, 4%, 3%, or no more than rRNA bases. In some instances sequencing results in 1-10%, 1-8%, 1-6%, 2-15%, 2-10%, 2-8%, 2-5%, or 3-5% rRNA bases. In some instances sequencing results in no more than 15%, 10%, 8%, 7%, 6%, 5%, 4%, 3%, or no more than rRNA bases without rRNA depletion. In some instances sequencing results in 1-10%, 1-8%, 1-6%, 2-15%, 2-10%, 2-8%, 2-5%, or 3-5% rRNA bases without rRNA depletion. In some instances sequencing results in at least 50%, 55%, 60%, 65%, 70%, 75%, 80%, 85%, 90%, or at least 95% expression profiling efficiency. In some instances sequencing results in 50-99%, 50-95%, 50-90%, 50-80%, 50-70%, 60-95%, 60-90%, 60-99%, 70-99%, 70-95%, 70-90%, 75-99%, 75-90%, 75-85%, 80-99%, 80-95%, or 90-99% expression profiling efficiency. In some instances sequencing results in no more than 10%, 9%, 8%, 7%, 6%, 5%, 4.5%, 4%, 3.5%, 3%, 2.5%, 2%, 1.5%, or no more than 1.2% duplication. In some instances sequencing results in no more than 0.5-10%, 0.5-5%, 0.5-3%, 0.5-2.5%, 0.5-2%, 0.5-1%, 1-3%, 1-2.5%, 1-5%, or 1.5-3% duplication. In some instances sequencing results in no more than 5%, 4.5%, 4%, 3.5%, 3%, 2.7%, 2.5%, 2.3%, 2%, 1.8%, 1.5%, 1.2%, 1%, 0.9%, 0.8%, 0.7%, 0.6%, or no more than 0.5% incorrect read strands. In some instances sequencing results in 0.1-5%, 0.1-4%, 0.1-3%, 0.1-2.5%, 0.1-2%, 0.1-1.8%, 0.1-1.5%, 0.1-1.2%, 0.1-1%, 0.5-2%, 0.5-1.5%, 0.5-1.8%, or 1-1.5% incorrect read strands. In some instances sequencing results in no more than 10%, 8%, 7%, 6%, 5.5%, 5%, 4.5%, 4%, 3%, 2.5%, 2%, 1.5%, 1%, 0.9%, 0.8%, 0.7%, or no more than 0.6% median 3′ bias. In some instances sequencing results in 0.1-5%, 0.1-4%, 0.1-3%, 0.1-2%, 0.1-1.5%, 0.1-1.2%, 0.1-1%, 0.1-0.8%, 0.1-0.7%, 0.1-0.6%, 0.1-0.5%, 0.5-1%, 0.5-0.8%, 0.5-3%, or 0.5-5% median 3′ bias. In some instances at least 10%, 20%, 30%, 40%, 50%, 60%, 70%, 80%, or at least 90% of sequenced bases are coding DNA sequences (CDS). In some instances 40-99%, 40-95%, 40-95%, 50-95%, 75-99%, 75-95%, 75-90% of sequenced bases are coding DNA sequences (CDS). In some instances, the amount of input cDNA is at least 1, 2, 3, 5, 7, 10, 12, 15, 20, 25, 50, 75, 100, 125, 150, or at least 175 ng. In some instances, the amount of input cDNA is 1-200, 1-150, 1-125, 1-100, 1-75, 1-50, 1-25, 5-50, 5-25, 10-150, 10-125, 25-200, 25-150, 50-150, 50-250, or 75-125 ng. Further provided herein are methods wherein sequencing comprises detection of at least one RNA fusion.

Downstream applications of polynucleotide libraries may include next generation sequencing. For example, enrichment of target sequences with a controlled stoichiometry polynucleotide probe library results in more efficient sequencing. The performance of a polynucleotide library for capturing or hybridizing to targets may be defined by a number of different metrics describing efficiency, accuracy, and precision. For example, Picard metrics comprise variables such as HS library size (the number of unique molecules in the library that correspond to target regions, calculated from read pairs), mean target coverage (the percentage of bases reaching a specific coverage level), depth of coverage (number of reads including a given nucleotide) fold enrichment (sequence reads mapping uniquely to the target/reads mapping to the total sample, multiplied by the total sample length/target length), percent off-bait bases (percent of bases not corresponding to bases of the probes/baits), usable bases on target, AT or GC dropout rate, fold 80 base penalty (fold over-coverage needed to raise 80 percent of non-zero targets to the mean coverage level), percent zero coverage targets, PF reads (the number of reads passing a quality filter), percent selected bases (the sum of on-bait bases and near-bait bases divided by the total aligned bases), percent duplication, or other variable consistent with the specification.

Read depth (sequencing depth, or sampling) represents the total number of times a sequenced nucleic acid fragment (a “read”) is obtained for a sequence. Theoretical read depth is defined as the expected number of times the same nucleotide is read, assuming reads are perfectly distributed throughout an idealized genome. Read depth is expressed as function of % coverage (or coverage breadth). For example, 10 million reads of a 1 million base genome, perfectly distributed, theoretically results in 10× read depth of 100% of the sequences. Experimentally, a greater number of reads (higher theoretical read depth, or oversampling) may be needed to obtain the desired read depth for a percentage of the target sequences. Enrichment of target sequences with a controlled stoichiometry probe library increases the efficiency of downstream sequencing, as fewer total reads will be required to obtain an experimental outcome with an acceptable number of reads over a desired % of target sequences. For example, in some instances 55× theoretical read depth of target sequences results in at least 30× coverage of at least 90% of the sequences. In some instances no more than 55× theoretical read depth of target sequences results in at least 30× read depth of at least 80% of the sequences. In some instances no more than 55× theoretical read depth of target sequences results in at least 30× read depth of at least 95% of the sequences. In some instances no more than 55× theoretical read depth of target sequences results in at least 10× read depth of at least 98% of the sequences. In some instances, 55× theoretical read depth of target sequences results in at least 20× read depth of at least 98% of the sequences. In some instances no more than 55× theoretical read depth of target sequences results in at least 5× read depth of at least 98% of the sequences. Increasing the concentration of probes during hybridization with targets can lead to an increase in read depth. In some instances, the concentration of probes is increased by at least 1.5×, 2.0×, 2.5×, 3×, 3.5×, 4×, 5×, or more than 5×. In some instances, increasing the probe concentration results in at least a 1000% increase, or a 20%, 30%, 40%, 50%, 60%, 70%, 80%, 90%, 100%, 200%, 300%, 500%, 750%, 1000%, or more than a 1000% increase in read depth. In some instances, increasing the probe concentration by 3× results in a 1000% increase in read depth.

On-target rate represents the percentage of sequencing reads that correspond with the desired target sequences. In some instances, a controlled stoichiometry polynucleotide probe library results in an on-target rate of at least 30%, or at least 35%, 40%, 45%, 50%, 55%, 60%, 65%, 70%, 75%, 80%, 85%, or at least 90%. Increasing the concentration of polynucleotide probes during contact with target nucleic acids leads to an increase in the on-target rate. In some instances, the concentration of probes is increased by at least 1.5×, 2.0×, 2.5×, 3×, 3.5×, 4×, 5×, or more than 5×. In some instances, increasing the probe concentration results in at least a 20% increase, or a 10%, 20%, 30%, 40%, 50%, 60%, 70%, 80%, 90%, 100%, 200%, 300%, or at least a 500% increase in on-target binding. In some instances, increasing the probe concentration by 3× results in a 20% increase in on-target rate.

Coverage uniformity is in some cases calculated as the read depth as a function of the target sequence identity. Higher coverage uniformity results in a lower number of sequencing reads needed to obtain the desired read depth. For example, a property of the target sequence may affect the read depth, for example, high or low GC or AT content, repeating sequences, trailing adenines, secondary structure, affinity for target sequence binding (for amplification, enrichment, or detection), stability, melting temperature, biological activity, ability to assemble into larger fragments, sequences containing modified nucleotides or nucleotide analogues, or any other property of polynucleotides. Enrichment of target sequences with controlled stoichiometry polynucleotide probe libraries results in higher coverage uniformity after sequencing. In some instances, 95% of the sequences have a read depth that is within 1× of the mean library read depth, or about 0.05, 0.1, 0.2, 0.5, 0.7, 1, 1.2, 1.5, 1.7 or about within 2× the mean library read depth. In some instances, 80%, 85%, 90%, 95%, 97%, or 99% of the sequences have a read depth that is within 1× of the mean.

Enrichment of Target Nucleic Acids with a Polynucleotide Probe Library

A probe library described herein may be used to enrich target polynucleotides present in a population of sample polynucleotides, for a variety of downstream applications. In one some instances, a sample is obtained from one or more sources, and the population of sample polynucleotides is isolated using conventional techniques known in the art. Samples are obtained (by way of non-limiting example) from biological sources such as saliva, blood, tissue, skin, or completely synthetic sources. The plurality of polynucleotides obtained from the sample are fragmented, end-repaired, and adenylated to form a double stranded sample nucleic acid fragment. In some instances, end repair is accomplished by treatment with one or more enzymes, such as T4 DNA polymerase, klenow enzyme, and T4 polynucleotide kinase in an appropriate buffer. A nucleotide overhang to facilitate ligation to adapters is added, in some instances with 3′ to 5′ exo minus klenow fragment and dATP.

Adapters may be ligated to both ends of the sample polynucleotide fragments with a ligase, such as T4 ligase, to produce a library of adapter-tagged polynucleotide strands, and the adapter-tagged polynucleotide library is amplified with primers, such as universal primers. In some instances, the adapters are Y-shaped adapters comprising one or more primer binding sites, one or more grafting regions, and one or more index regions. In some instances, the one or more index region is present on each strand of the adapter. In some instances, grafting regions are complementary to a flowcell surface, and facilitate next generation sequencing of sample libraries. In some instances, Y-shaped adapters comprise partially complementary sequences. In some instances, Y-shaped adapters comprise a single thymidine overhang which hybridizes to the overhanging adenine of the double stranded adapter-tagged polynucleotide strands. Y-shaped adapters may comprise modified nucleic acids, that are resistant to cleavage. For example, a phosphorothioate backbone is used to attach an overhanging thymidine to the 3′ end of the adapters. The library of double stranded sample nucleic acid fragments is then denatured in the presence of adapter blockers. Adapter blockers minimize off-target hybridization of probes to the adapter sequences (instead of target sequences) present on the adapter-tagged polynucleotide strands. Denaturation is carried out in some instances at 96° C., or at about 85, 87, 90, 92, 95, 97, 98 or about 99° C. A polynucleotide targeting library (probe library) is denatured in a hybridization solution, in some instances at 96° C., at about 85, 87, 90, 92, 95, 97, 98 or 99° C. The denatured adapter-tagged polynucleotide library and the hybridization solution are incubated for a suitable amount of time and at a suitable temperature to allow the probes to hybridize with their complementary target sequences. In some instances, a suitable hybridization temperature is about 45 to 80° C., or at least 45, 50, 55, 60, 65, 70, 75, 80, 85, or 90° C. In some instances, the hybridization temperature is 70° C. In some instances, a suitable hybridization time is 16 hours, or at least 4, 6, 8, 10, 12, 14, 16, 18, 20, 22, or more than 22 hours, or about 12 to 20 hours. Binding buffer is then added to the hybridized adapter-tagged-polynucleotide probes, and a solid support comprising a capture moiety are used to selectively bind the hybridized adapter-tagged polynucleotide-probes. The solid support is washed with buffer to remove unbound polynucleotides before an elution buffer is added to release the enriched, tagged polynucleotide fragments from the solid support. In some instances, the solid support is washed 2 times, or 1, 2, 3, 4, 5, or 6 times. The enriched library of adapter-tagged polynucleotide fragments is amplified and the enriched library is sequenced.

A plurality of nucleic acids (i.e. genomic sequence) may obtained from a sample, and fragmented, optionally end-repaired, and adenylated. Adapters are ligated to both ends of the polynucleotide fragments to produce a library of adapter-tagged polynucleotide strands, and the adapter-tagged polynucleotide library is amplified. The adapter-tagged polynucleotide library is then denatured at high temperature, preferably 96° C., in the presence of adapter blockers. A polynucleotide targeting library (probe library) is denatured in a hybridization solution at high temperature, preferably about 90 to 99° C., and combined with the denatured, tagged polynucleotide library in hybridization solution for about 10 to 24 hours at about 45 to 80° C. Binding buffer is then added to the hybridized tagged polynucleotide probes, and a solid support comprising a capture moiety are used to selectively bind the hybridized adapter-tagged polynucleotide-probes. The solid support is washed one or more times with buffer, preferably about 2 and 5 times to remove unbound polynucleotides before an elution buffer is added to release the enriched, adapter-tagged polynucleotide fragments from the solid support. The enriched library of adapter-tagged polynucleotide fragments is amplified and then the library is sequenced. Alternative experimental variables such as incubation times, temperatures, reaction volumes/concentrations, number of washes, or other variables consistent with the specification are also employed in the method.

A population of polynucleotides may be enriched prior to adapter ligation. In one example, a plurality of polynucleotides is obtained from a sample, fragmented, optionally end-repaired, and denatured at high temperature, preferably 90-99° C. A polynucleotide targeting library (probe library) is denatured in a hybridization solution at high temperature, preferably about 90 to 99° C., and combined with the denatured, tagged polynucleotide library in hybridization solution for about 10 to 24 hours at about 45 to 80° C. Binding buffer is then added to the hybridized tagged polynucleotide probes, and a solid support comprising a capture moiety are used to selectively bind the hybridized adapter-tagged polynucleotide-probes. The solid support is washed one or more times with buffer, preferably about 2 and 5 times to remove unbound polynucleotides before an elution buffer is added to release the enriched, adapter-tagged polynucleotide fragments from the solid support. The enriched polynucleotide fragments are then polyadenylated, adapters are ligated to both ends of the polynucleotide fragments to produce a library of adapter-tagged polynucleotide strands, and the adapter-tagged polynucleotide library is amplified. The adapter-tagged polynucleotide library is then sequenced.

A polynucleotide targeting library may also be used to filter undesired sequences from a plurality of polynucleotides, by hybridizing to undesired fragments. For example, a plurality of polynucleotides is obtained from a sample, and fragmented, optionally end-repaired, and adenylated. Adapters are ligated to both ends of the polynucleotide fragments to produce a library of adapter-tagged polynucleotide strands, and the adapter-tagged polynucleotide library is amplified. Alternatively, adenylation and adapter ligation steps are instead performed after enrichment of the sample polynucleotides. The adapter-tagged polynucleotide library is then denatured at high temperature, preferably 90-99° C., in the presence of adapter blockers. A polynucleotide filtering library (probe library) designed to remove undesired, non-target sequences is denatured in a hybridization solution at high temperature, preferably about 90 to 99° C., and combined with the denatured, tagged polynucleotide library in hybridization solution for about 10 to 24 hours at about 45 to 80° C. Binding buffer is then added to the hybridized tagged polynucleotide probes, and a solid support comprising a capture moiety are used to selectively bind the hybridized adapter-tagged polynucleotide-probes. The solid support is washed one or more times with buffer, preferably about 1 and 5 times to elute unbound adapter-tagged polynucleotide fragments. The enriched library of unbound adapter-tagged polynucleotide fragments is amplified and then the amplified library is sequenced.

Provided herein are synthetic polynucleotide libraries comprising a plurality of polynucleotides, wherein the polynucleotides comprise DNA, wherein the polynucleotides are configured to hybridize with one or more exon regions of target nucleic acids comprising RNA. In some instances, the polynucleotides are 80-160 bases in length. In some instances, the library comprises at least 50,000 polynucleotides. In some instances, the library comprises 100,000 to 750,000 polynucleotides. In some instances, the exon regions encode for at least 500 genes. In some instances, a portion of the genes comprise two or more isoforms. The In some instances, the library further comprises the plurality of target nucleic acids. In some instances, at least a portion of the polynucleotides is biotinylated. In some instances, the library is configured to minimize hybridization with housekeeping genes. In some instances, housekeeping genes comprise the highest 1.5% expressed genes in a cell. In some instances, the cell is human. In some instances, the stoichiometry of the plurality of polynucleotides is adjusted based on mRNA transcript abundance. In some instances, the polynucleotides are tiled over the one or more exon regions. In some instances, library hybridization bias is minimized towards one or more exon-exon junctions.

Provided herein are methods for sequencing comprising contacting a library described herein with a sample comprising a plurality of target nucleic acids, wherein the plurality of target nucleic acids comprises RNA; enriching at least one nucleic acid that binds to the library; and sequencing the at least one enriched target nucleic acid.

### Examples

The following examples are set forth to illustrate more clearly the principles and practice of embodiments disclosed herein to those skilled in the art and are not to be construed as limiting the scope of any claimed embodiments.

### Example 1: Preliminary RNA Exome Design

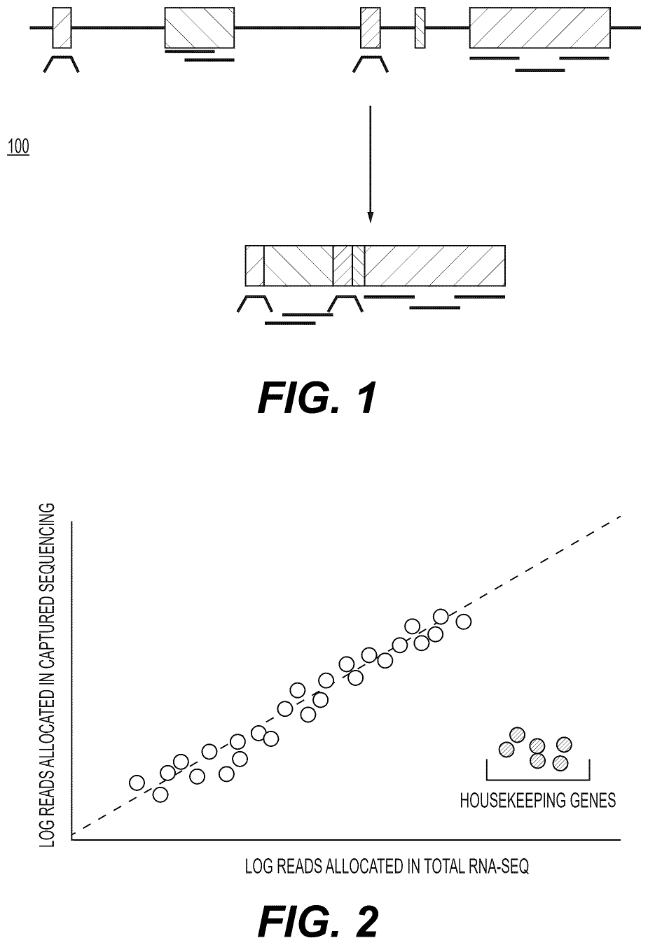

A process was designed for RNA capture panels. The primary goal was to avoid bias in capturing different isoforms (or novel fusions) (FIG. 1). Exons longer than probe length were tiled end-to-end and exons between ½ probe length and full probe length were printed mismatches at ends. Exons less than 40 nt relied on shadow capture to cover.

One opportunity to improve capture of low-expressed transcripts was to remove (or reduce coverage of) housekeeping genes (FIG. 2). Based on tissue-specific GTEx expression data in humans, it appeared that taking out the top 1% of genes made read depths 1.6-5 fold higher over the low-expressed transcripts.

The oncology panels were designed where targets were defined by CDS's (not UTRs) defined in GenCode v39. All CDS's listed in all isoforms in GenCode were merged together and genes were taken from (1) 800 kb cancer panel (to have a general survey of oncology targets), (2) genes from the RNA fusion standards product, (3) genes from Taniue K and Akemitsu N, 2021, incorporated herein by reference in its entirety, for canonical fusion drivers, and (4) genes from Heyer, E E et al (2019), incorporated herein by reference in its entirety, describing an RNA fusion detection panel. The content of the oncology panel was trimmed to avoid high-expression genes without a very strong role in cancer. In total, the merged targets occupied about 1.38 Mbp of space on the genome.

The oncology panels targeted 1×-tiled using a designer code. Sequences were fetched from DNA using designer. Two versions of panel were designed-one with DNA sequence, one “masked”. The masked panel included regions outside of target on the probe were replaced by a random AT-rich (˜25% GC) sequence. In some instances, target may be placed at one end of probe. The panels were designed to avoid biasing towards capture of any contaminating DNA. Additionally, targets less than or equal to 40 bp were excluded.

The oncology panels were designed using BLAT matches against hg39 transcript sequences (including non-coding) to reduce off-target binding. The off-target risk was designed using relative expression (mean of GTEX). For example, if target gene A has expression E_{A}, define off-target risk as ΣEᵢ/E_{A}, e.g., the total capture of all off-target regions vs the target region. Probes were kept where “off-target risk” was less than 10 (98.8% of total probes). This meat that at least 10% of the reads from this probe were expected to derive from the expected target.

The overall coverage of relevant transcripts were assessed. Using the design strategy (e.g., no filtering) and 15 bp slop, 99.8% of total bases were covered among all listed exons. Over each transcript, >95% coverage over was achieved in all but one (510/511) transcripts. With off-target filtering and 15 bp slop, <95% coverage was achieved over 492/511 genes over targeted transcripts. Of these transcripts, none seem necessary to cover 100%. Small enough list to manually curate. Over all listed transcripts, 99.3% of total bases were covered.

RNA capture strategy was then designed, as shown in FIG. 3. For a transcript, as shown in FIG. 3, the design included two probes, including one directly targeting the known splice variant, For a fusion of a gene at an exon 1 junction, the design included only one probe. This strategy guaranteed at least one probe will target fusions (FIGS. 4A and 4B).

One design goal included excluding highly-expressed transcripts. In some instances, isolating gene sets could allow significant read savings (e.g., 2- to 5-fold depending on tissue for top 1% of genes). This could be roughly 520 genes by GTEx's definition. In some instances, a set of removed genes needed curation. Several considerations for panel design included how deep to go into different isoforms, coverage of UTRs, handling of off-targets, inclusion of regions with short exons (e.g., less than 20 base pairs).

### Example 2: RNA Exome Design Development

The capture strategy and panels as generally designed according to Example 1 were further developed. Panels were mapped against CCDS and total coverage was investigated. A class of genes (e.g., polymorphic pseudogenes) were identified as potentially genes to cover. Panels were also compared against hg38 mapping positions with those used in Illumina's exome, as well as further exome targets (minus UTRs and intergenic regions). The updates to the design included targeting 19728 protein-coding genes and 30268 transcripts. Attempts were made to cover these genes with an “exon-aware” strategy, such that bias is minimized towards particular exon-exon junctions. The panels were split into two sub-panels by expression. The first sub-panel was for high-expression genes, which were for genes in the top 1% of mean expression among all tissues in GTEx, and probes with significant off-target in these transcripts (8057 probes total). The second sub-panel was for core genes in the lower 99% of genes by mean expression in GTEx (419327 unique probes).

The testing strategy included UHR makes for a low-expression panel alone, combined panel, and combined panel with partial biotin for high expression genes, which could be used to establish splice-site awareness (with OEM data). In some instances, the testing strategy comprises a differential expression system. In some instances, the testing strategy comprises profiling success at detecting fusions (e.g., fusion event in UHR, RNA fusion standard, etc.).

Designs were further revised. Revisions included a more encompassing design of transcript variants, switching to 80 bp probes instead of 120 for increased flexibility, isolating true “housekeeping” genes rather than highly-expressed genes (e.g., relatively constant expression). Further investigation also included the question for capture uniformity vs accurate expression. Switching to 80 bp probes comprised using 70 bp as the largest exon with exon-aware probes. The strategy for selecting transcripts was also changed from originally selecting exons based on CCDS with at least one transcript for every protein-coding gene, prioritizing well-annotated transcript models to covering all transcripts that are annotated as a part of Gencode Basic. As a result, the probes went from 427k to 602k probes. For 80 bp alone, it was expected to be about 534k probes. The housekeeping genes were picked from those in top 1.5% of transcripts (mean >146 TPM) where CV (stdev/mean) across tissues is less than 90%. Some “housekeeping” genes ranked on these metrics shown below in Table 1. In total, 355 genes were selected.

TABLE 1

| Gene symbol | Mean expression | CV for expression |
|---|---|---|
| ACTB | 3464.59 | 88% |
| AHSP | 3.46 | 643% |
| B2M | 1101.36 | 83% |
| GAPDH | 1309.36 | 84% |
| HBS1L | 8.61 | 39% |
| HPRT1 | 34.10 | 77% |
| SDHA | 102.25 | 47% |
| TBP | 15.51 | 54% |

99.64% of all transcripts in CCDS were covered by probes. 18/18773 (<0.1%) of genes were covered over <95% of the coding sequence on average among all transcripts. Gaps in coverage appeared to be mostly due to probes that were removed for homology to RNA genes (e.g., rRNA).

Exon-aware tradeoff were also investigated (FIGS. 5A-5B). It was determined that probes were not evenly tiled across transcripts and had to be placed with higher density near short exons. This led to discrepancies in probe density across the transcript, which could complicate expression analysis.

The development was further focused on splice variant discovery. Trial prints were tested for 80 bp vs 120 bp, and printing ˜10% of gene loci, including genes in the planned RNA fusion standards which were selected evenly across ranks of expression. ERCC standards were included as well. Panels were experimentally compared as evidence for preferring one or the other strategy.

A first experiment was set up with the goal of using exome V2 in hybrid capture using RNAseq library using WM Depletion and RNAseq kits as a reference point before finalizing the RNA exome print. The experiment investigated how read depth across different transcripts compared to an uncaptured RNA-seq, such as whether/how capture re-shapes detection compared to expression, and in particular results across some of highly expressed transcripts, as well as how much the uniformity across each transcript is affected by the apparent tiling.

The Library Conditions included: 100 ng UHR input, Two operators (DC+KB), WM Depletion and RNAseq, Mass input: 50 ng, 100 ng, 500 ng, 1000 ng, Adapter input: 2.5 ul and 5 ul, and Cycling: 10 cycles. The Capture Conditions included: Exome V2, ST V2 Capture Protocol, and NextSeq 550 2×74 bp. The wetlab and sequencing results are provided in Table 2. Here, DNA Libraries made at 50 ng of gDNA into a library preparation protocol and 200 ng and 500 ng into TE were used as controls.

TABLE 2

| | | | Final | | | |
|---|---|---|---|---|---|---|
| Lib Mass | | | Concentration | Average | Loading | |
| Input:TE | Adapter | | (Qubit, | Frag | Concentration::PhiX | Sequencer::Cluster |
| Mass Input | Input | | ng/μL) | Size | into Sequencing | Density::Q30::PF |
| 50 ng::200 ng | 2.5 | μL | 15.85 | 348 | 1.8 pM::5% | NextSeq |
| 100 ng::200 ng | 2.5 | μL | 15.55 | 356 | | #7::~280::92.24%::83.77% |
| 500 ng::500 ng | 5 | μL | 23.4 | 383 | | |
| 1000 ng::500 ng | 5 | μL | 23.5 | 369 | | |
| 500 ng::500 ng | 2.5 | μL | 20.15 | 374 | | |
| 1000 ng::500 ng | 2.5 | μL | 18.8 | 376 | | |

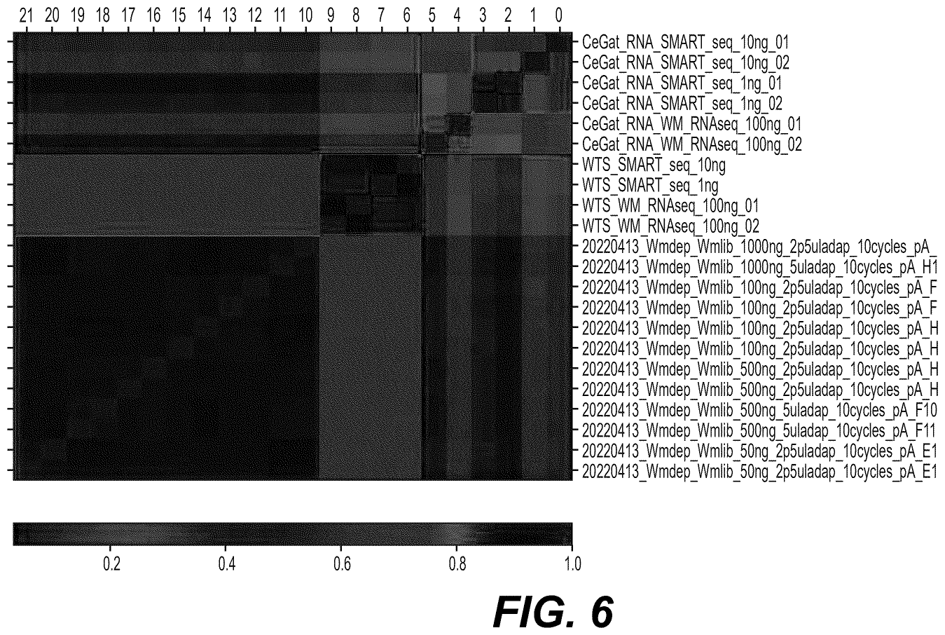

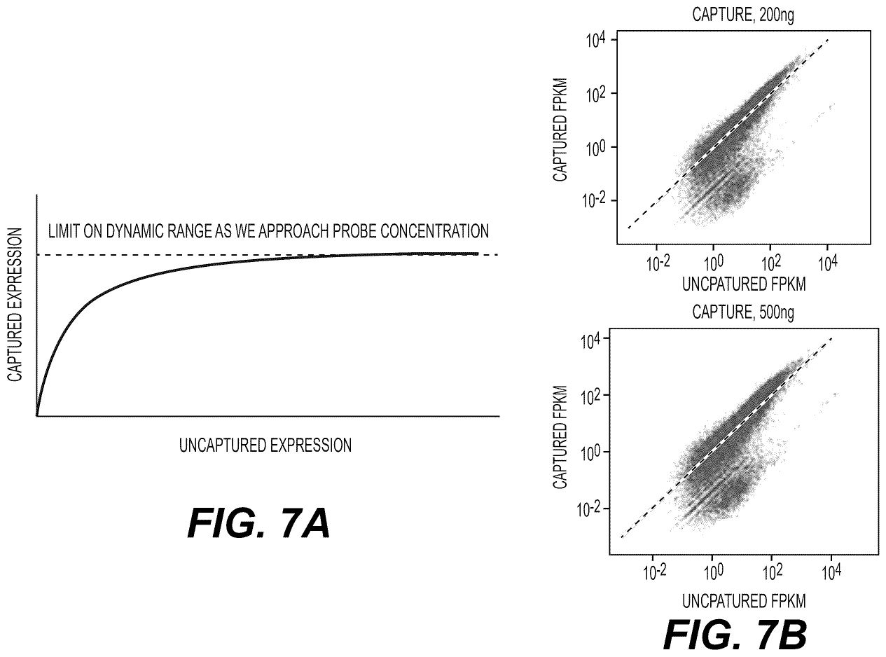

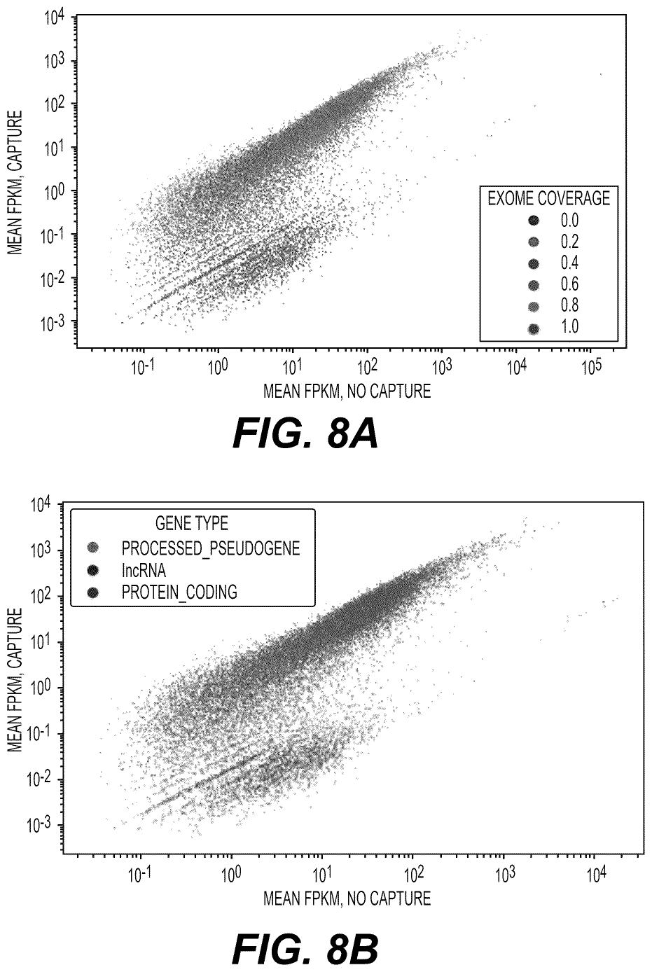

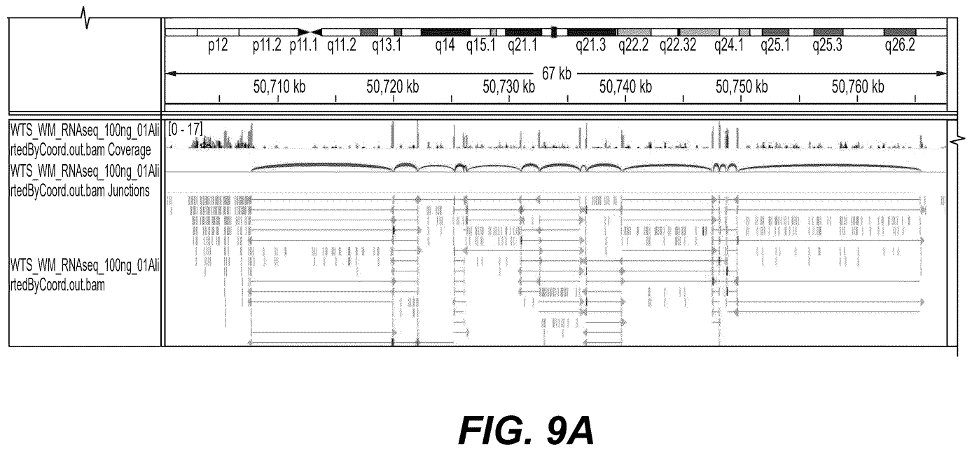

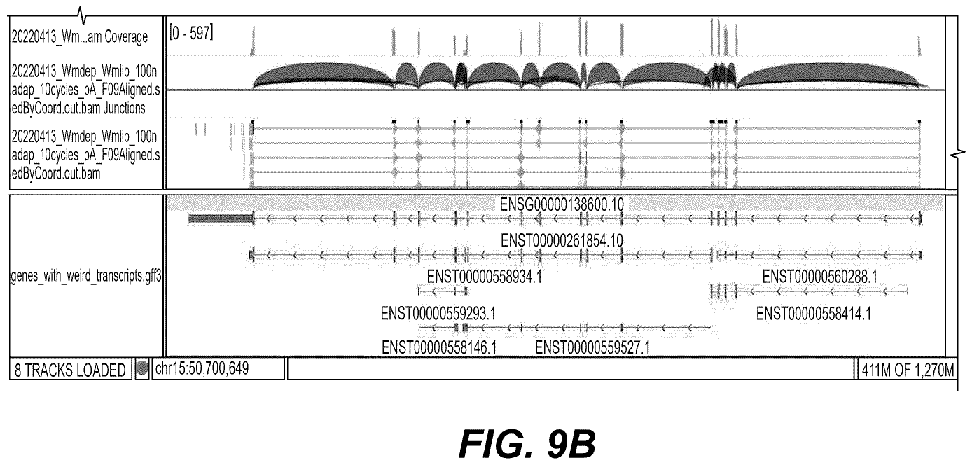

FIG. 6 provides a heatmap showing an overall sample correlation matrix. WTS or Exome captures did not correlate within a block. WTS correlated generally well with the exome, and somewhat well between conditions. Exome captures correlated well with each other. FIGS. 7A and 7B further shows expectations vs. reality of the capture, where an overall improvement in capture of 1.4-fold was achieved. It was also identified that uncaptured region were primarily non-target regions (FIGS. 8A and 8B). An initial look at splice-variant bias (FIGS. 9A and 9B) indicated many examples of extreme bias in capture, only targeting CDS, so differences in UTR length appeared to massively change outcomes.

### Example 3: RNA Exome Panel Proof of Concept

The WM Depletion and RNAseq Kit with hybrid capture was used with the RNA Fusion panel and compared to the Takara single cell kit using the same panel as a proof of concept. This was done using 10 ng and 1 ng of RNA input. A schematic of the depletion and RNAseq kit is provided in FIG. 10 and a RNAseq workflow is provided in FIG. 11.

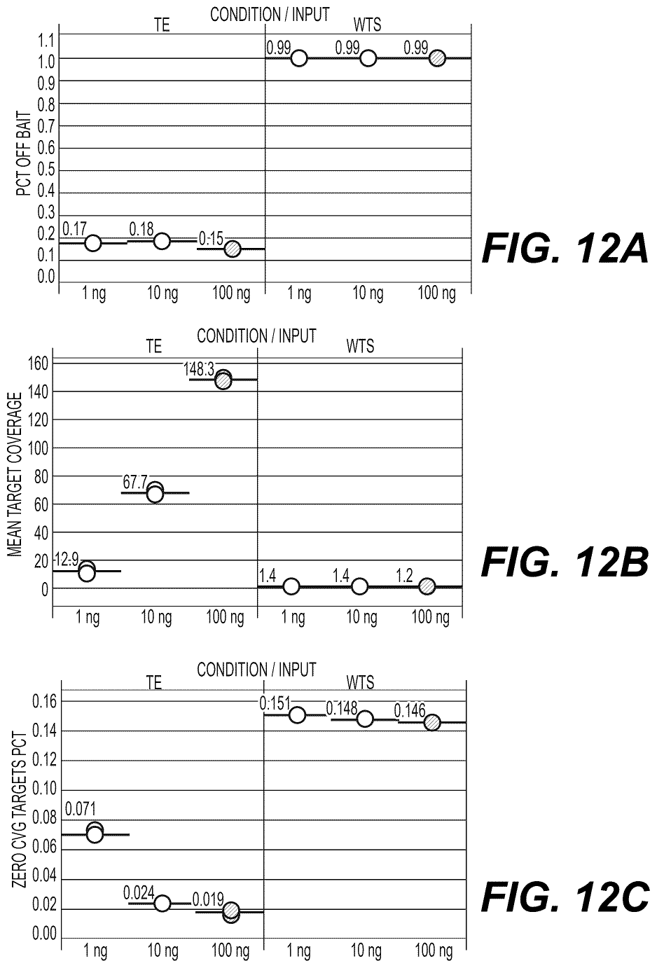

RNA libraries were generated using two different kits. The first was the Takara SMART Seq, where two experimental conditions were performed: (1) 1 ng input-PCR1 at 5 cycles, PCR2 at 15 cycles; and (2) 10 ng input-PCR1 at 5 cycles, PCR2 at 13 cycles. The second was WM RNAseq Kit with 100 ng input-10 cycles. Duplicate captures were performed for each kit and input level using STv2 and sequencing was done on a Nextseq550 with 2×76 bp sequencing. WTS was also performed. Results are provided in FIGS. 12A-12C.

Updates were made to the bioinformatic pipeline and target list ambiguities. The target list did not contain genomic coordinates, rather synthetic contigs of junction sequence were created and spiked into reference. These 90 junctions were unlikely to exist in UHR material. Additionally, as working solution, targets were defined as the genomic positions of the gene (entire pre-mRNA transcript from 5′-3′ UTR including intronic sequences) with a total of 46 genes, including intronic sequences. QC metrics calculated before gene expression quantification were also made the same regardless of target genes. Further steps were added to produce a filtered GTF containing all elements attributed to the target genes.

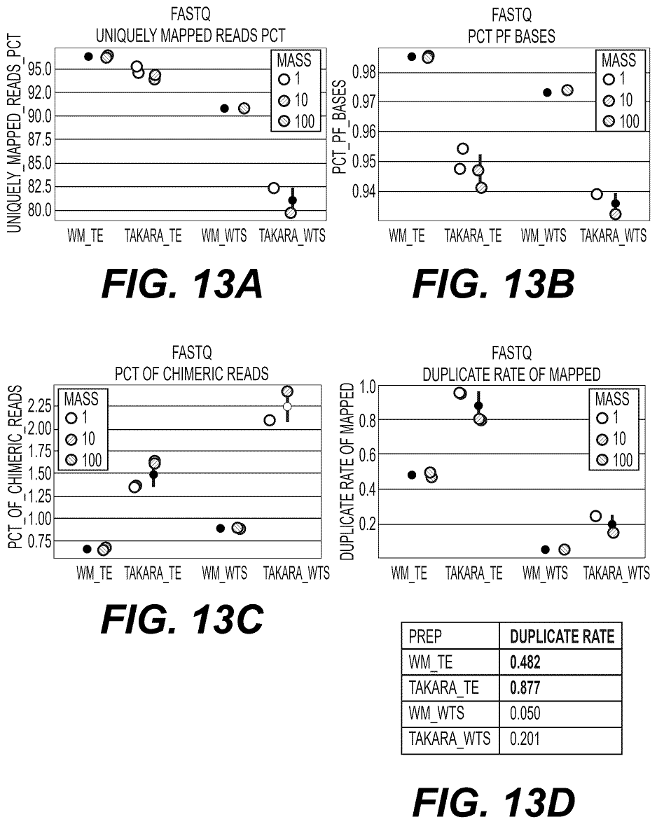

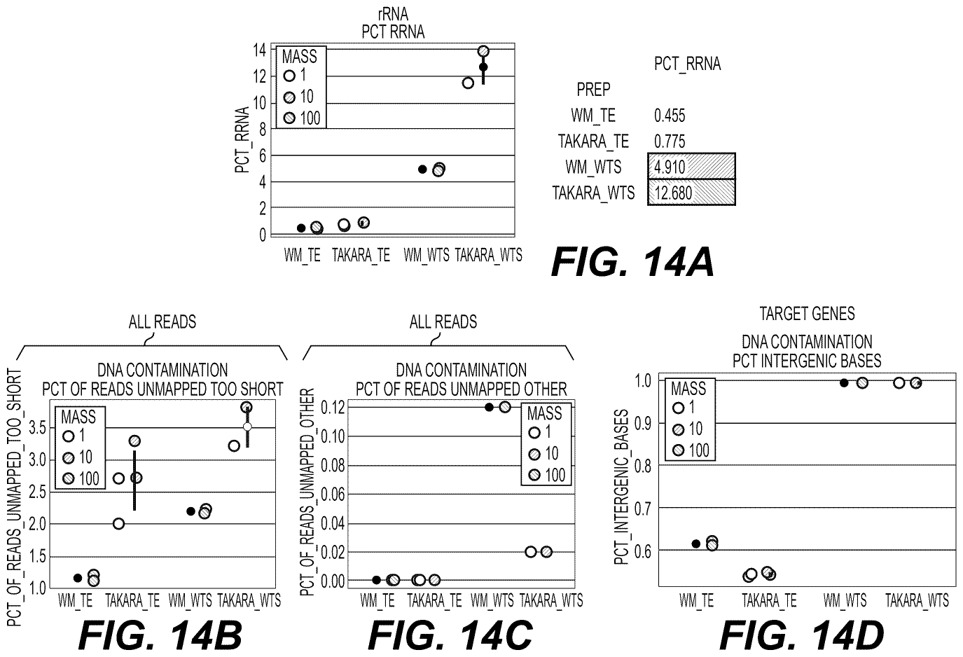

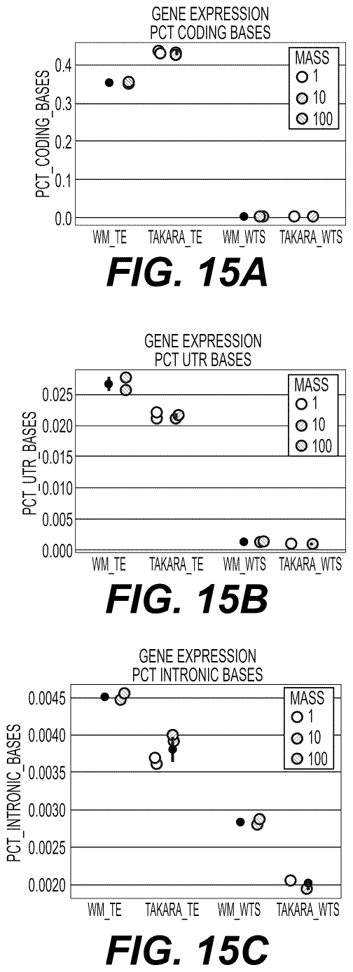

The TE resulted in a high burden of duplicate reads (FIGS. 13A-13D), where WM TE performed well with highest rates of uniquely mapped reads, PF bases, and low rate of chimeric reads. TE as a whole has a much higher duplicate rate, driven by mass input. Additionally, TE had a much lower rate of rRNA reads (FIGS. 14A-14D), where WM WTS had expected ˜5% rRNA abundance. It was expected to see lower rRNA rates for TE. Takara TE had a wide variation of reads unmapped too short, which was not necessarily contam. WM TE had slightly higher intergenic rate near target genes. Furthermore, WM TE sequenced more UTR than Takara (FIGS. 15A-C). It was expect to see bad performance for WTS. Metrics were restricted to target genes. A higher intronic burden in WM was still seen.

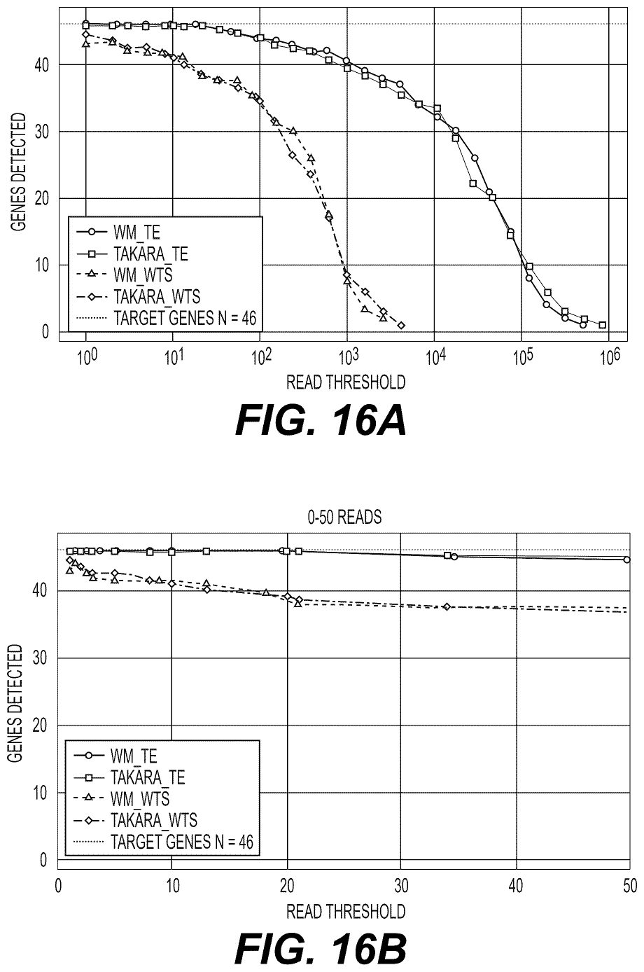

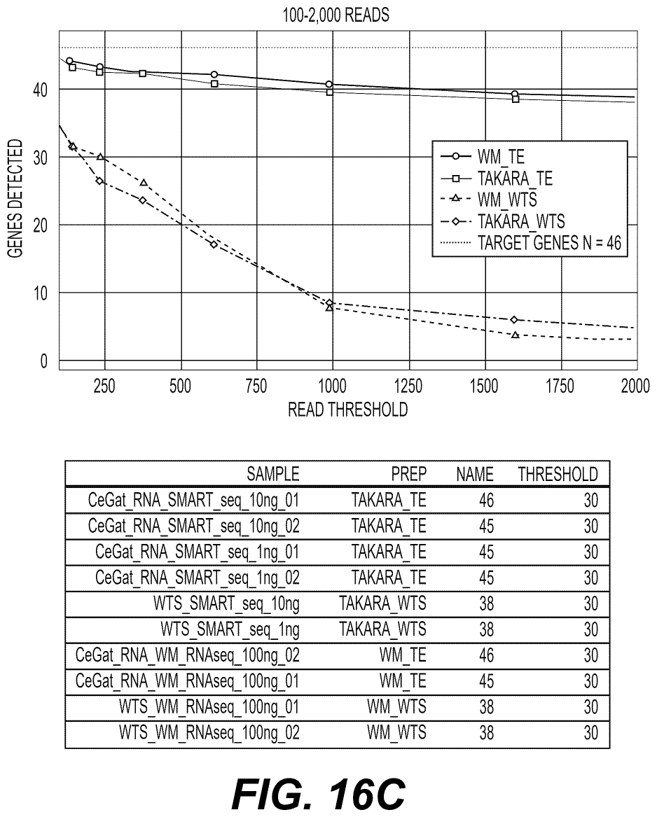

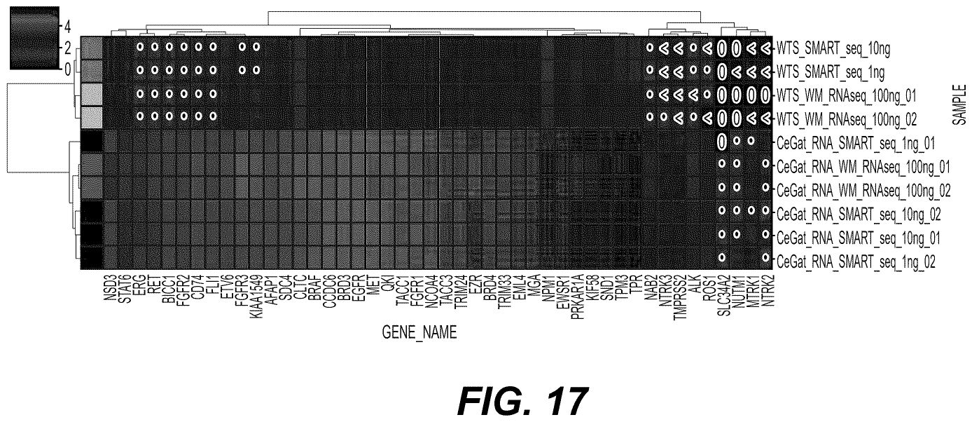

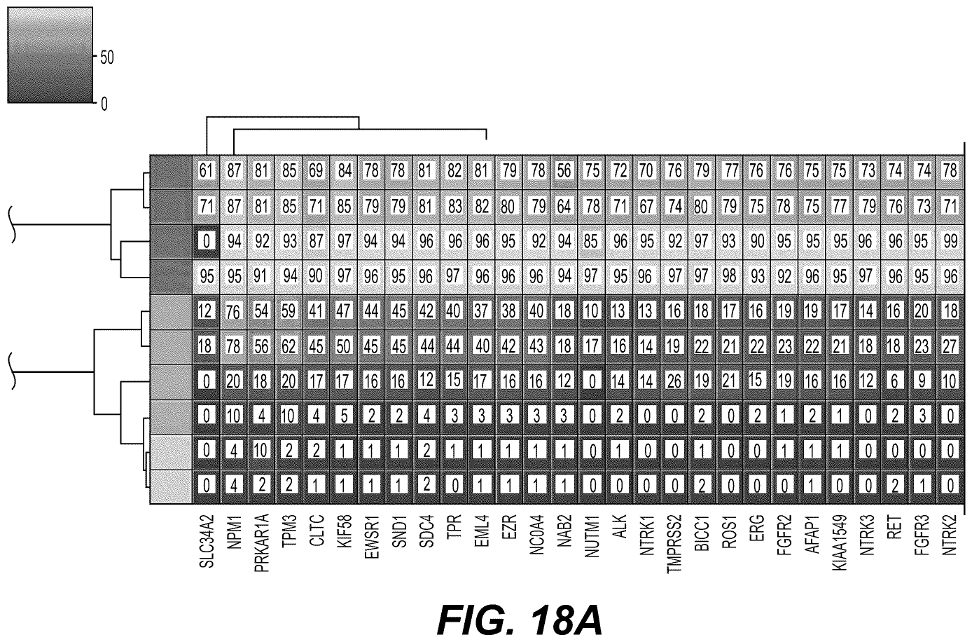

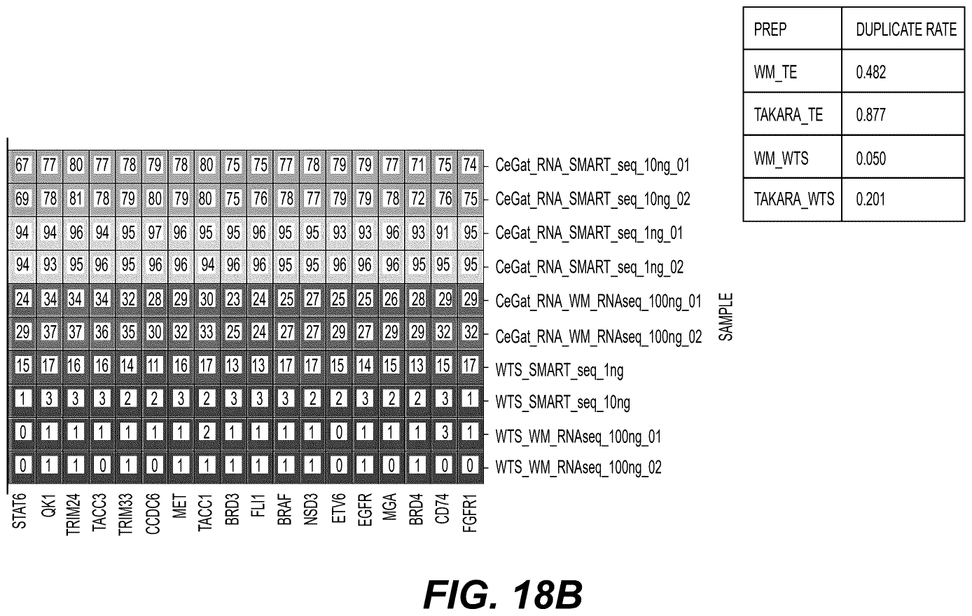

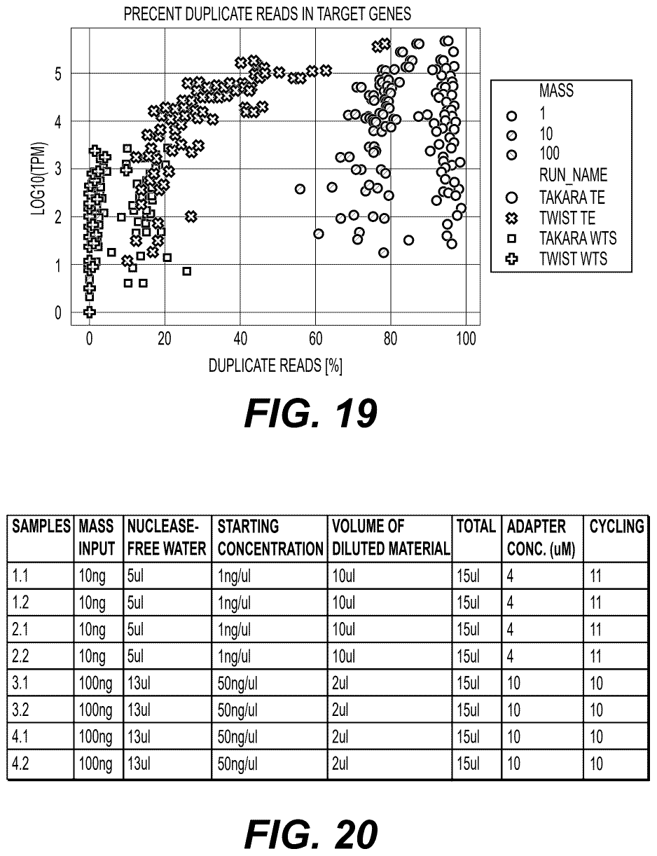

It was also shown that TE captured more target gene sequence (FIGS. 16A-16C). Here, the TE was prominent, and both WM and Takara TE were similar. Around 30× was when dropouts of genes began to appear. TE captures lowly expressed genes 1-2 orders of magnitude greater than WTS (FIG. 17) and TE has a higher duplicate read rate (FIGS. 18A and 18B). This showed duplicate rate was correlated to the input mass. Expression and read duplicate Rates were also correlated for higher mass TE (FIG. 19).

Since previous data showed that diversity seemed to be kit specific when using lower mass inputs, several different RNAseq kits were investigated and ran through capture using the RNA Fusion Panel. A goal was to use the WM Beta, NEB, and NEB+RNAseq Kit with hybrid capture using the RNA Fusion panel and compare it to previous data. This was done using 100 ng, 10 ng and Ing of RNA input. The experimental details are provided in Table 3.

TABLE 3

| RNAseq | Adapter | | Average Final Concentration | |
|---|---|---|---|---|
| Kit Name | Volumes | Cycling | when using 100 ng | |
| WM Beta | 1 ng-1 μL | 1 ng-13 | 110.5 +/− 6.36 | ng/μL |
| | 10 ng-2 μL | 10 ng-11 | | |
| | 100 ng-5 μL | 100 ng-10 | | |
| NEB | 1 ng-2.5 μL | 1 ng-17 | 129.43 +/− 60.99 | ng/μL |
| | 10 ng-2.5 μL | 10 ng-15 | | |
| | 100 ng-2.5 μL | 100 ng-12 | | |
| NEB + | 1 ng-2.5 μL | 1 ng-17 | 199.17 +/− 44.34 | ng/μL |
| RNAseq | 10 ng-2.5 μL | 10 ng-15 | | |
| Kit | 100 ng-2.5 μL | 100 ng-12 | | |

### Example 4: Panel Design Testing 1

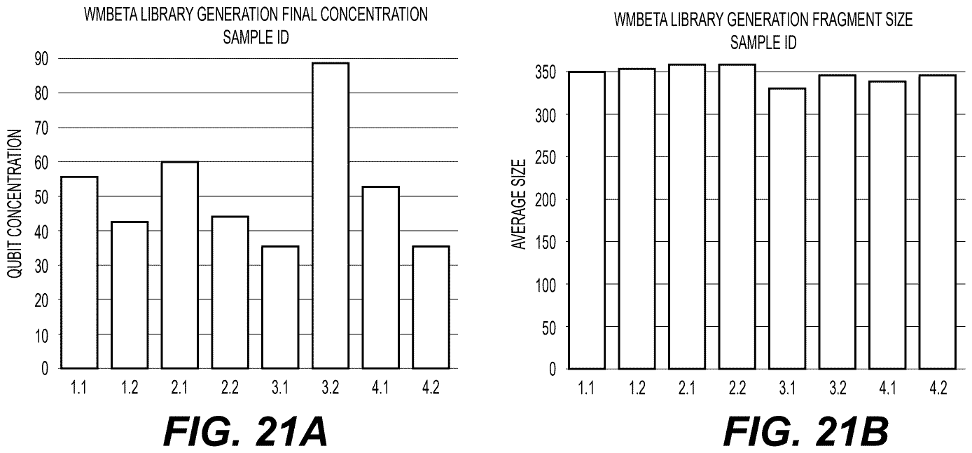

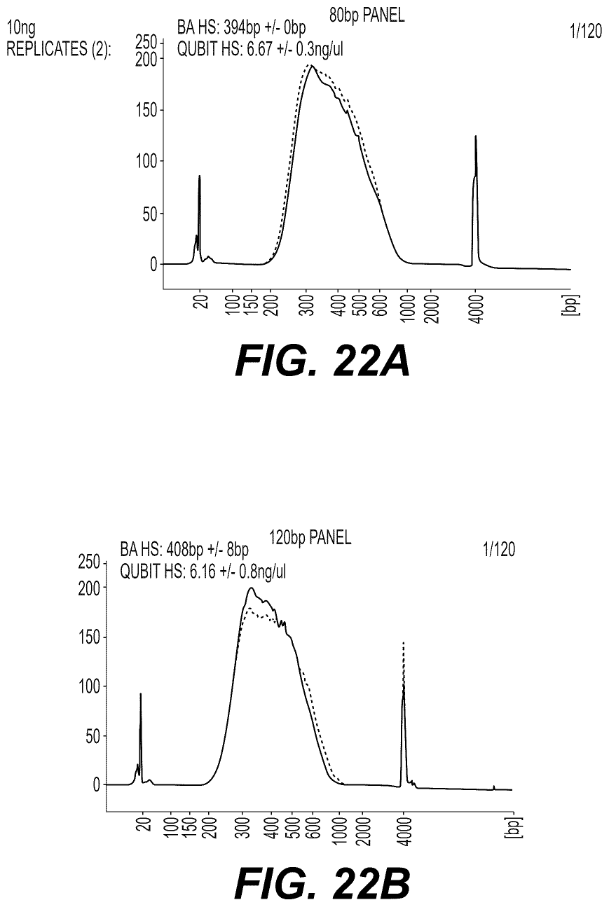

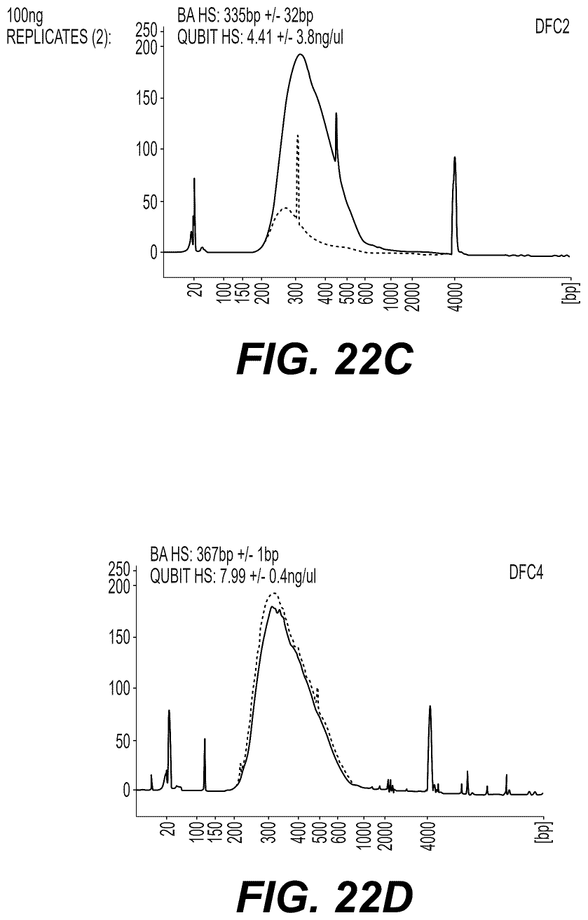

Based on the design considerations and results generally provided in Example 1-3, the following panel was designed: Alien-masked RNA Oncology Panel, Subset of the RNA Exome Panel using 120 bp probes vs 80 bp probes, and Top 1.5% housekeeping genes (to avoid having all transcripts detected be housekeeping genes). The library generation for 80 vs 120 bp testing is provided FIG. 20 and the library QC is provided in FIGS. 21A-21B. Capture and final QC of the panels are further provided in FIG. 22.

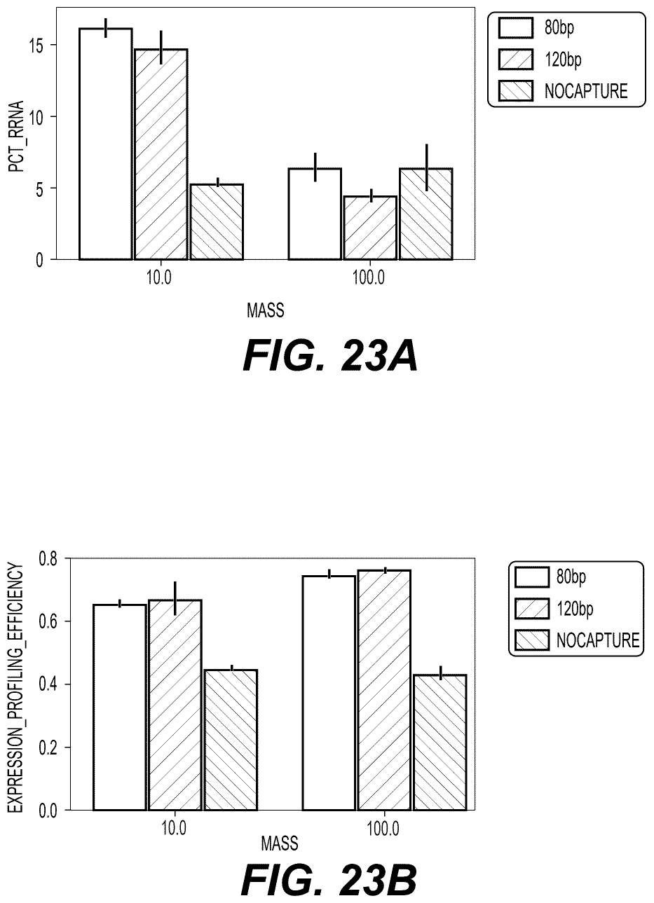

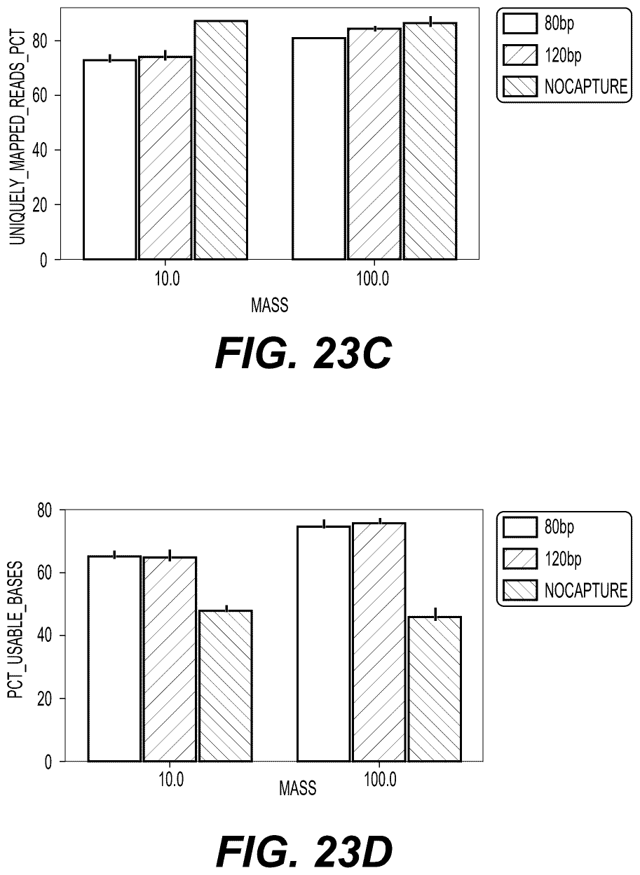

RNAseq metrics were further assessed (FIG. 23), which generally showed similar performance between 120 bp and 80 bp panels in terms of selecting bases from exons, which could be seen in expression_profiling_efficiency and pct_usable_base. There were some slight differences in total library complexity (80 bp is slightly lower). This could have been due to a small increase in the total amount of reads mapping to ribosomal elements in the 80 bp panel compared to the 120 bp panel.

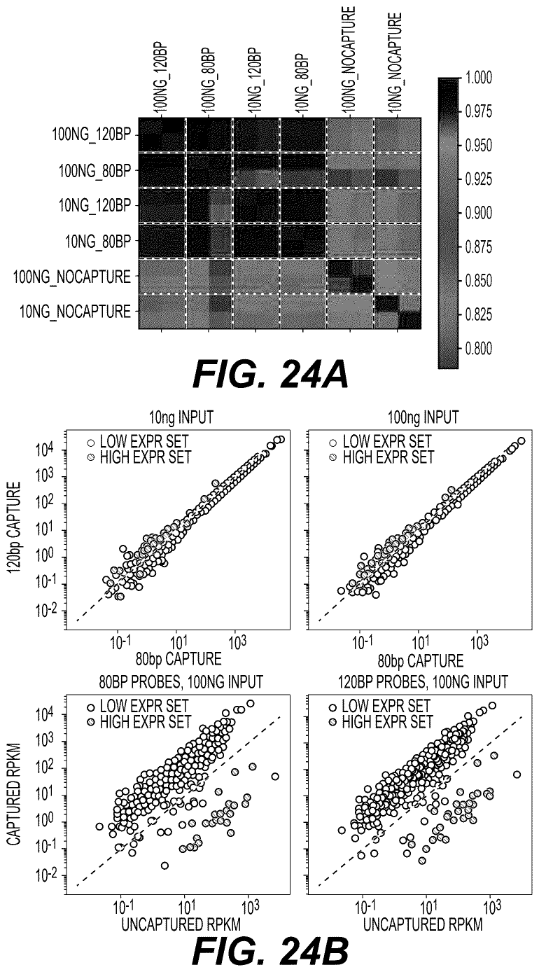

Expression levels were compared (FIGS. 24A-24B) which showed reproducible capture between different capture conditions. Generally similar trends were observed to what was observed with exomes. High expression probes were selected using GTEx data, but did not seem to be the highest expression genes in this dataset.

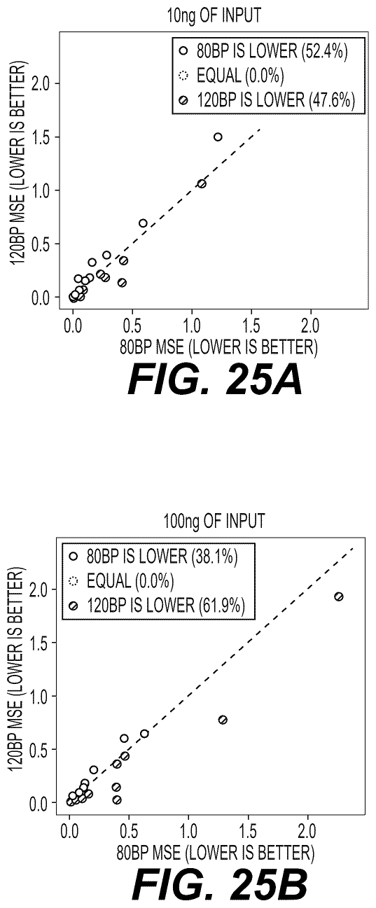

Isoform quantification biases was performed (FIGS. 25A and 25B), which was done using Salmon to obtain isoform-specific expression counts. Using these results genes were filtered with detectable differences in multiple targeted isoforms (21 genes total). Each transcript count was normalized out to the mean for the associated gene. Mean-squared error was calculated for the measurements in the 120 bp and 80 bp panels compared to uncaptured. Results did not appear to show a consistent benefit of 80 vs 120 bp, however, with a limited set of genes.

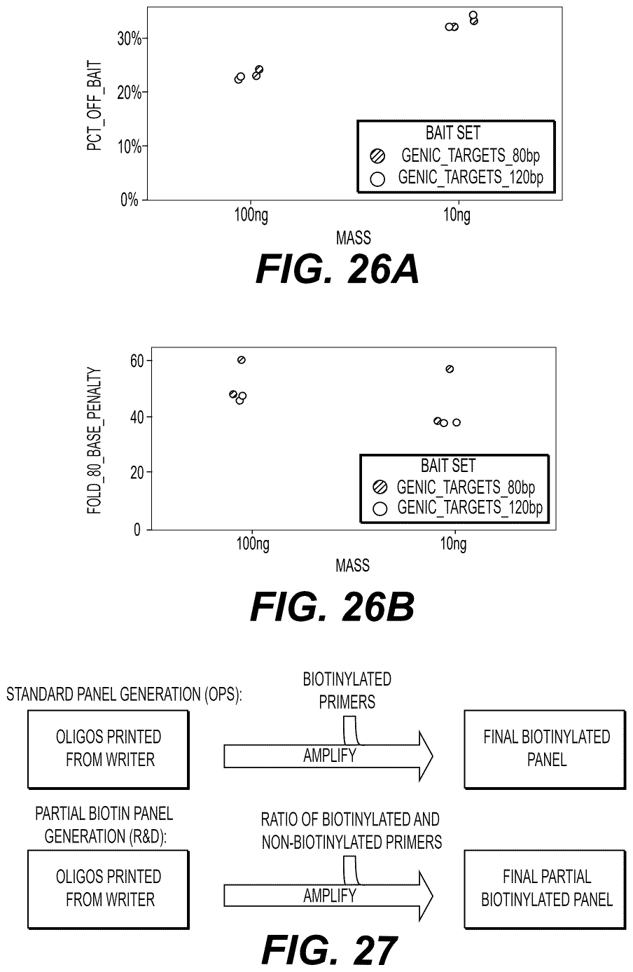

Capture results are further shown in FIGS. 26A and 26B in the DNA space. Capture was run both against transcript sequences (with exact probes) and hg38 (with estimated targets). Off-target was very high for RNA-space alignment, which may have been due to unincluded transcript variants (e.g., non-coding). PCT_OFF_BAIT in DNA-space was similar for 80 vs 120 bp probes. FOLD-80 seemed to be somewhat higher for the 80 bp probes.

Further considerations in the capture pipeline included results aligned both to transcripts and to DNA space, as well as understanding sources of intergenic signal in RNAseq-QC. Additionally, considerations included getting equivalents for off-target and fold-enrichment over the targets and for uniformity (e.g., fold-80 like metric for calculating and normalized per-transcript).

### Example 5: Panel Design Testing 2

Based on the results provided generally in Example 1-4, panels with 120 bp were selected for further development and the following panel was designed: Alien-masked RNA Oncology Panel; Subset of the RNA Exome Panel using 120 bp probes vs 80 bp probes; Top 1.5% housekeeping genes (to avoid having all transcripts detected be housekeeping genes).

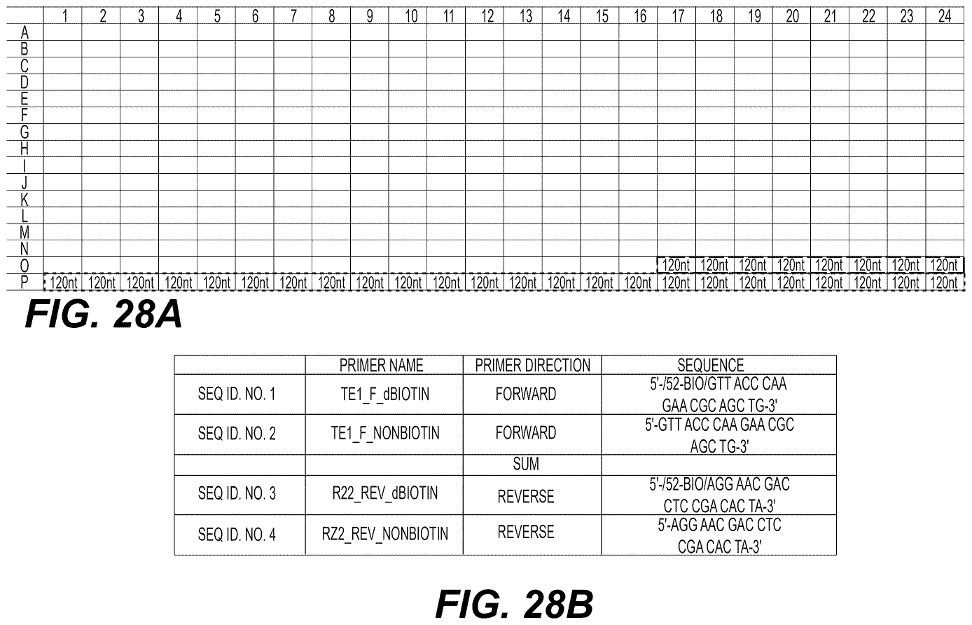

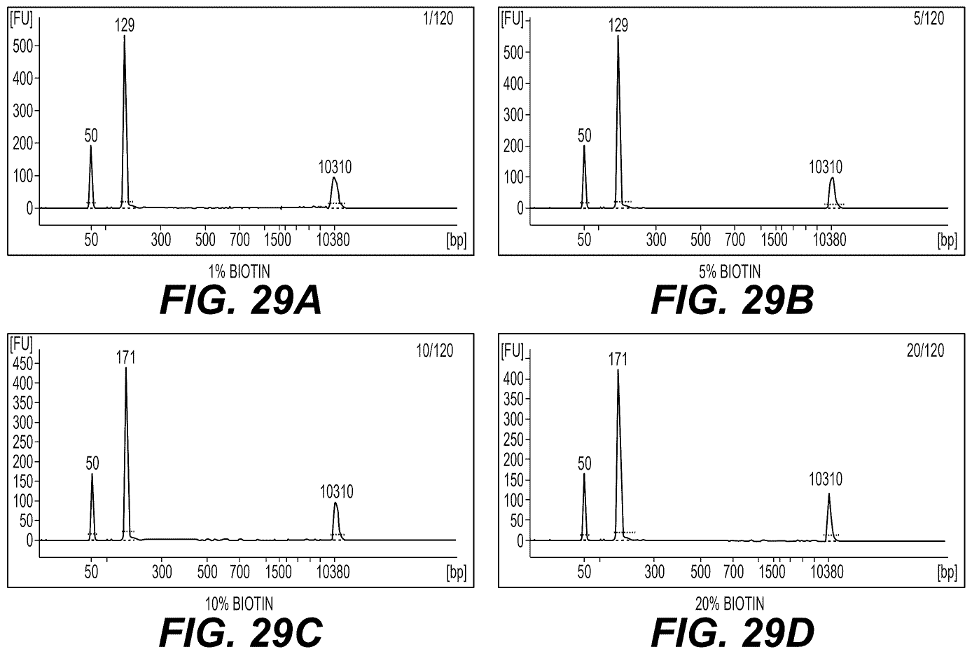

A general housekeeping gene detection scheme using biotin was designed in order to minimize the detection of such housekeeping genes (FIG. 27). A partial biotin panel was generated with the dilution plate and primers used (SEQ ID. NOs. 1-4) shown in FIGS. 28A and 28B, where the partial biotin primer ratios investigated include 1%, 5%, 10%, 20%, and 100%. The partial biotin panel was analyzed using an bioanalyzer for QC (FIGS. 29A-29D).

Two methods for biotin QC were developed. One using supernatant of streptavidin bead clean-up with the follow characteristics: Streptavidin bead clean-up using all ratios, Minelute column, QC using Qubit and Bioanalyzer, Remaining mass should not include biotin. The other methods used a biotin quantification kit with the following characteristics: HABA dye and avidin mix is added to the panel, and Biotin displaces HABA and changes absorbance.

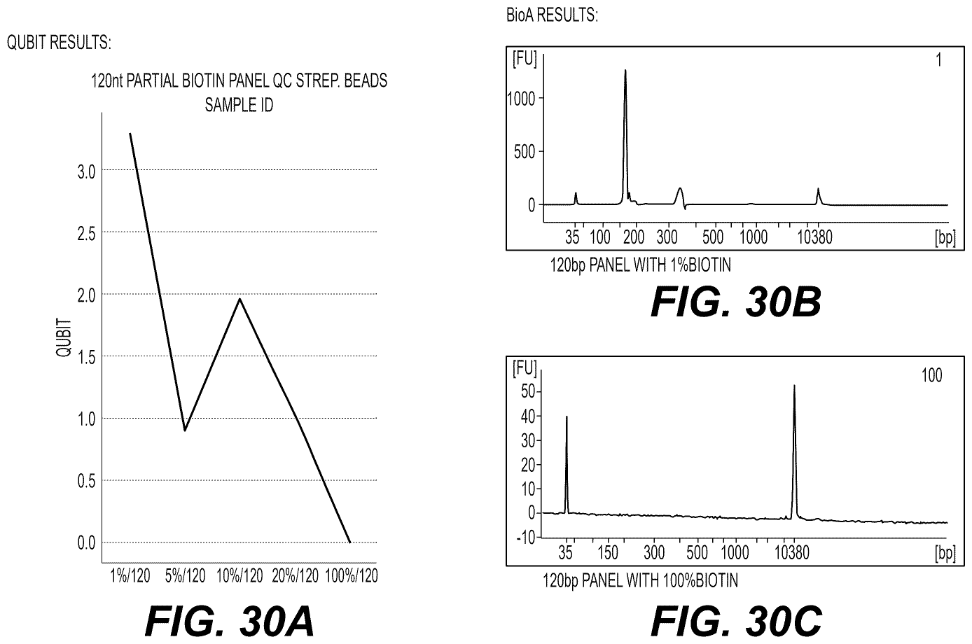

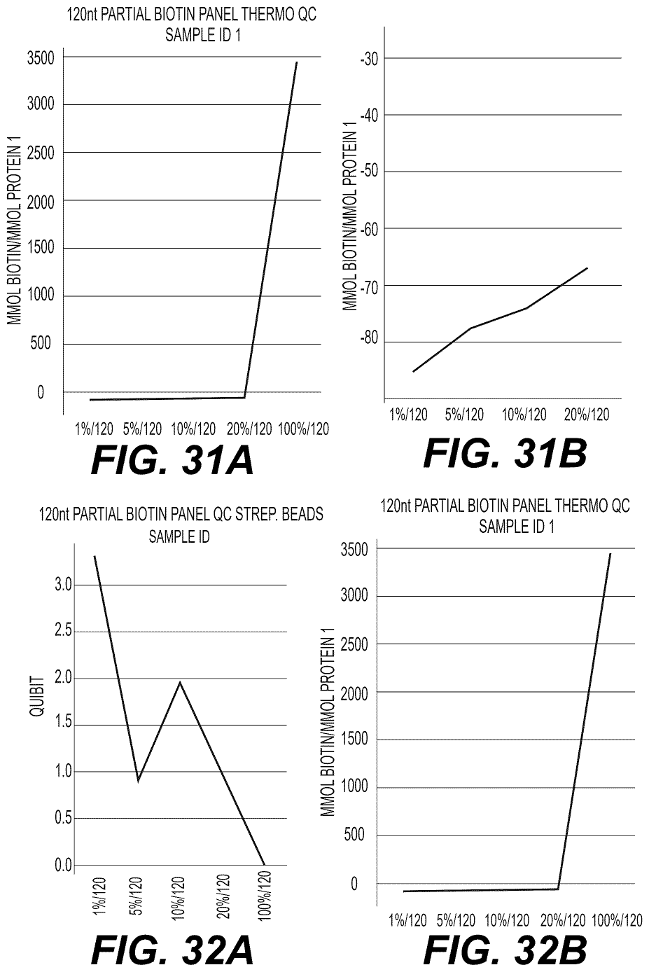

The results from the streptavidin bead clean up method is provided in FIGS. 30A-30C and the results from the biotin quantification kit are provided in FIGS. 31A and 31B. Upon QC comparison (FIGS. 32A and 32B), both methods showed noticeable differences between 100% biotinylated panels and partial biotin panels, which indicated both methods could be used for QC. Both streptavidin beads method and biotin quantification kit provided similar results/trend, suggesting similar performance. Outlier (10% biotin) in the streptavidin beads method may have been in part due to factors including poor mixing before Qubit, uneven beads distribution, or Qubit HS kit sensitivity. Based on these results, general QC recommendations are provided in Table 4.

TABLE 4

| Streptavidin Beads Method | Biotin Quantitation Kit |
|---|---|
| Longer, more complicated | Shorter, easier experiment (30 |
| experiment (~3 hrs) | mins to 1 hr) |
| Data interpretation is easier | Harder data interpretation (Would |
| (Qubit and BioA) | require decent calculation, but |
| | could be lessened by excel |
| | worksheet) |
| Can use current workflow with | Would require external resources |
| minimum modification | |

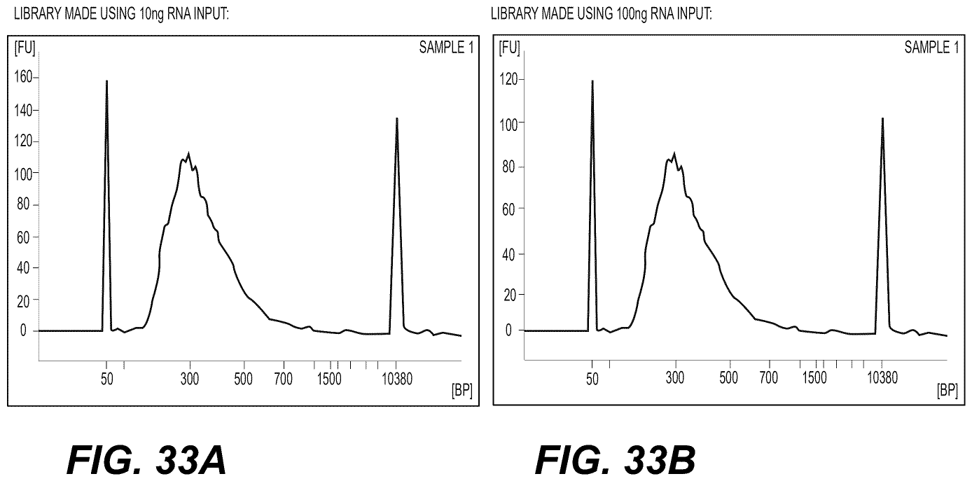

Additionally, partial biotin spike-in testing was performed to determine what percentage of partial biotin spike-in panel works best for keeping expression levels for housekeeping genes low but detectable (FIG. 33). Libraries were tested using 10 ng and 100 ng of input with UHR and ERCC, and using STv2 Capture protocol with 4 ul of partially biotinylated panels at 0.2 fmol/reaction/probe as spike-in and 4 ul of subset panel, all at 120 bp length: 1%, 5%, 10%, 20%, and 100%.

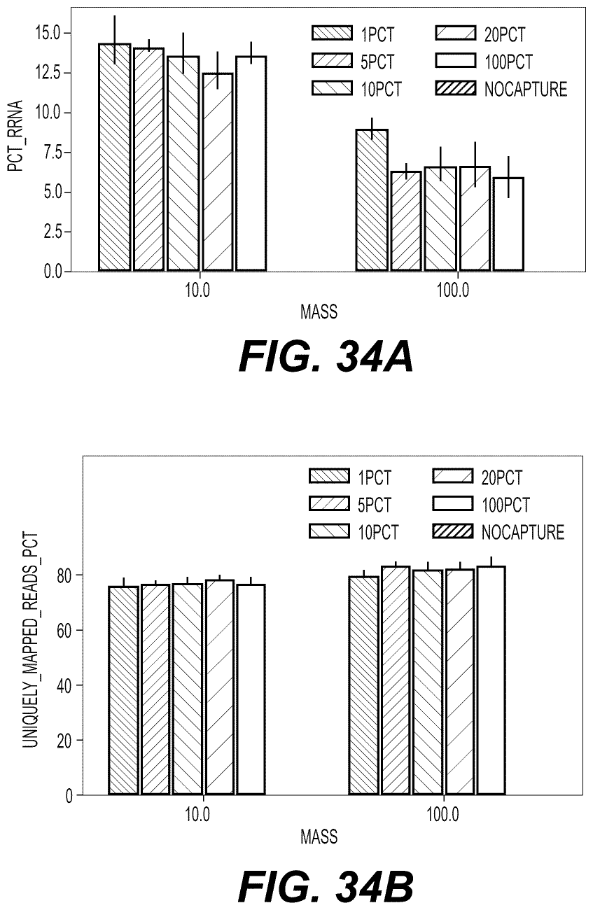

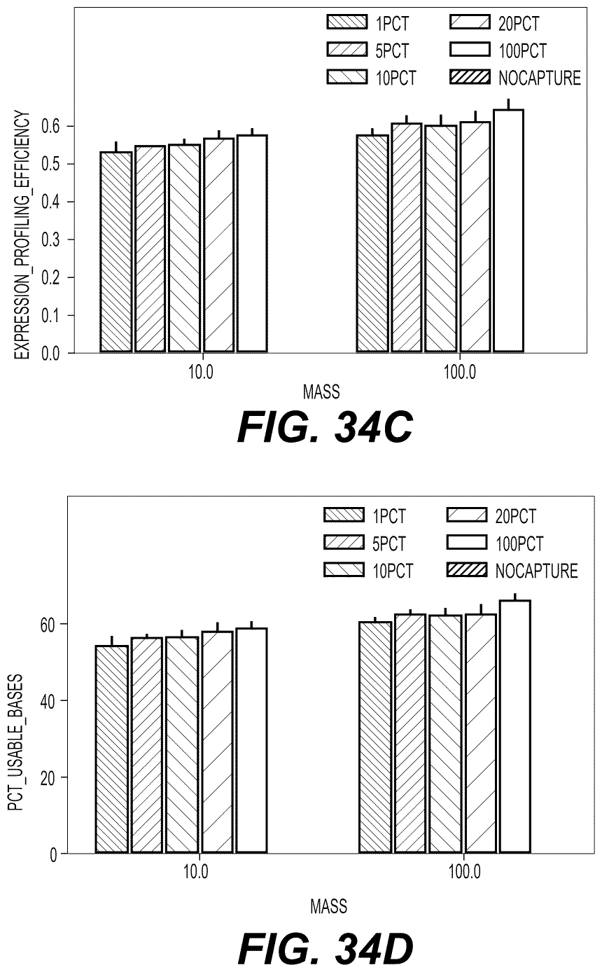

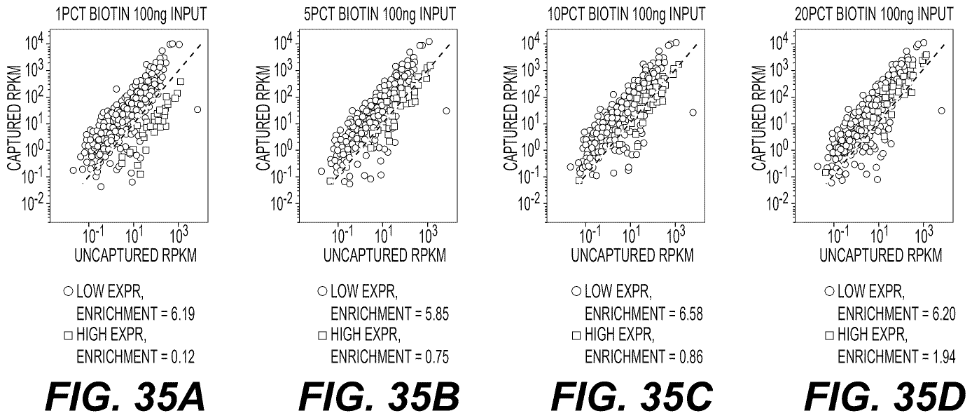

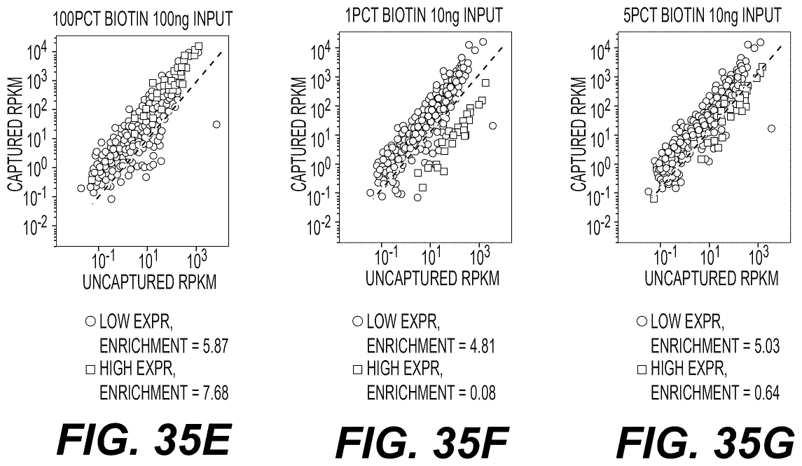

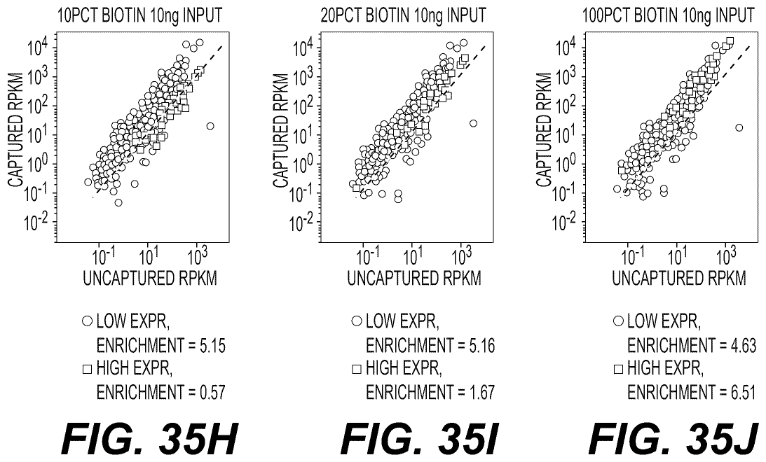

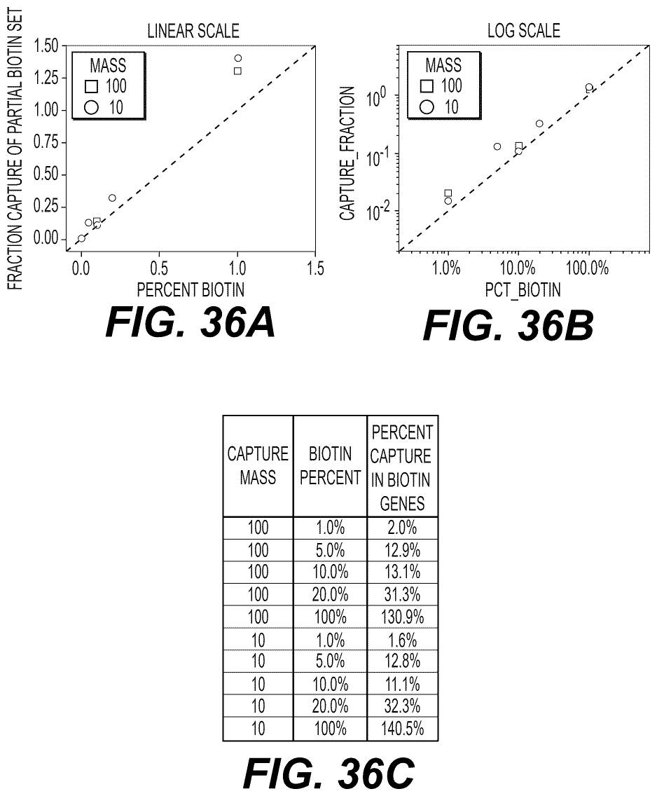

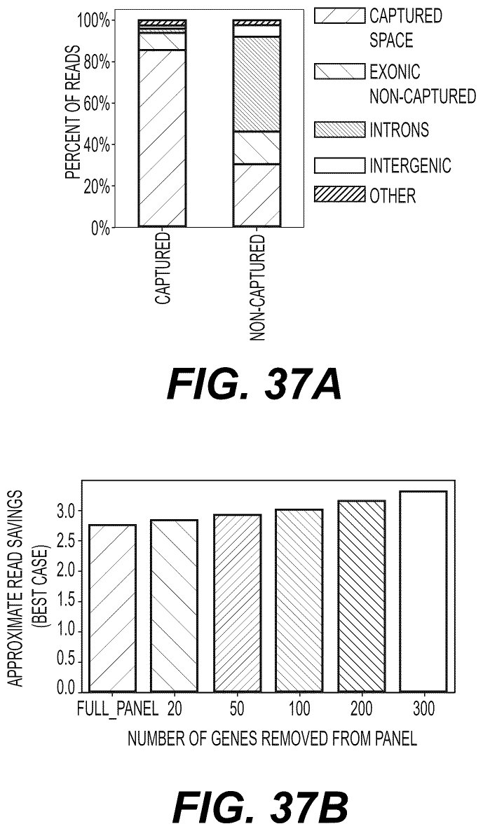

Overall metrics were assessed (FIGS. 34A-34D) with slightly more favorable metrics observed in terms of usable bases for higher mass percent of biotin. Correlation between captured and uncaptured were also investigated (FIGS. 35A-35J). Partial biotin vs standard subsets were further compared (FIGS. 36A-C). Comparison of the enrichment of non-biotin/biotin genes revealed some amazing agreement between the capture fraction and the input quantity of biotin. About 5% biotin sample seemed to be a little anomalously high, which may have been due to processing. Percent of reads in captured vs non-captured and the approximate read savings as the number of genes removed from a panel increases were finally determined (FIGS. 37A and 37B). Read savings from highly expressed genes showed marginal improvements compared to savings from excluding intron-containing reads, and reads from non-coding transcripts. Relatively marginal benefits were obtained from trimming a large number of genes (about 2.7-fold with no partial biotin, about 3.1-fold with removal of top 300 protein-coding genes).

A potential panel design for further investigation includes: Alien-masked RNA Oncology Panel, Subset of the RNA Exome Panel using 120 bp probes vs 80 bp probes, and Top 1.5% housekeeping genes—to avoid having all transcripts detected be housekeeping genes.

### Example 6: RNA Exome Panel for RNA Fusion Detection

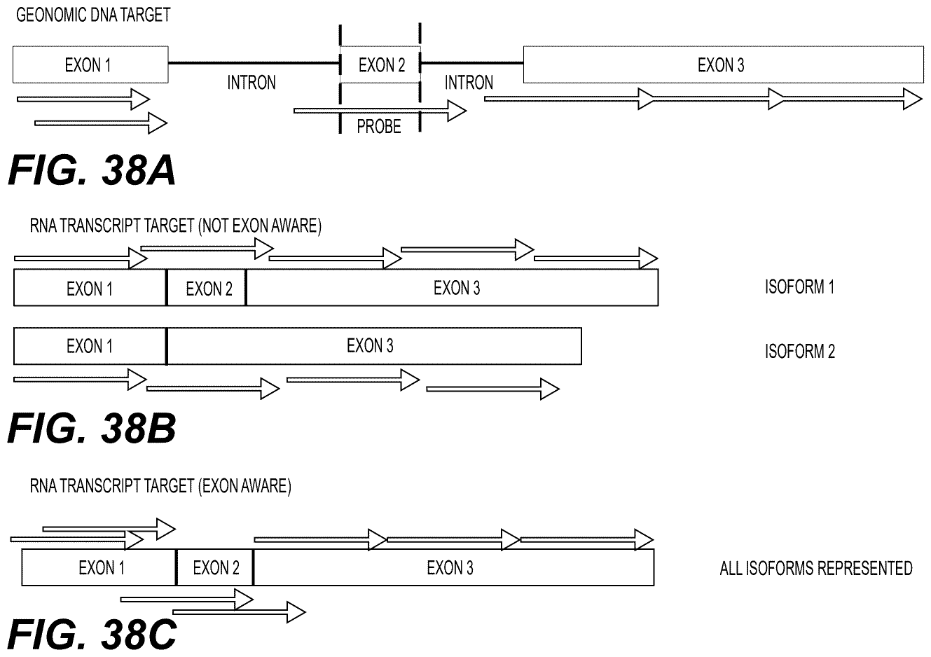

Total RNA sequencing provides a relatively unbiased view of the transcriptional state of a population of cells. However, many total RNA-seq experiments contend with a large number of reads that are not helpful for gene-expression analysis, including reads from highly abundant non-coding transcripts (like the 7SK RNA or ribosomal RNA), intronic reads from pre-mRNA, or contaminating genomic DNA. Target enrichment provides a way to focus sequencing on the informative parts of the genome, allowing for more sensitive detection of low-abundance transcripts, or for profiling only specific genes of interest. This example presents capture sequencing experiments using an RNA Exome panel described herein which uses a design strategy to specifically target every protein-coding isoform in Gencode v41 Basic. Although the design natively targets the transcriptome, the design strategy also places probes to minimize bias towards known isoforms and allow for discovery of isoforms or fusion genes (FIGS. 38A-38C). Panel performance in expression quantification was evaluated, showing that relative transcript abundances are preserved after hybrid capture. In some instances this allows for accurate and reproducible quantification of transcripts that are present across many orders of magnitude, as well as gains in sequencing efficiency from this targeted approach and demonstrate the ability to capture novel structural variants, such as RNA fusions common in cancers.

Design strategy and content selection. The first step in generating the RNA exome panel (or library) was to design both a content curation strategy and capture probe strategy against a transcript. Content curation was performed using the GenCode gene definitions (v41 on hg38), with a focus on the coding regions of protein-coding genes. To this end, the total defined CDS space was pared down in GenCode to categories of genes that were either protein-coding or with strong evidence for coding content in certain situations (see FIG. 39A). From these genes, a set of well-described transcript models was tiled, with the aim of natively covering the majority of isoforms that are of general interest to most researchers. For tiling strategies, three possibilities were evaluated: first, tiling the probes against content using the same strategy used for most DNA exomes (FIG. 39B). In some cases this has the advantage of being conceptually simple and handling multiple isoforms with a minimum of redundancy, but it may selectively capture gDNA and pre-mRNA, as it contains exon-intron junctions. Second, a straightforward tiling to the mature transcripts (FIG. 39C) was evaluated but it was found that this had probe redundancy and would likely select against novel isoforms or fusion transcripts, as it contains probes that span exon-exon boundaries. Finally, a design placing probes such that every exon-exon boundary contained at least one non-spanning probe (FIG. 39D) was evaluated. This reduced the number of distinct and redundant probes, avoided capturing intronic content, and avoided introducing additional bias towards content already represented in the design. This “exon-aware” design strategy was moved forward for production. After tiling the design using the exon-aware strategy above, exact duplicate probes were collapsed and low-sequence complexity and/or homology towards non-coding RNAs probes were removed that would reduce sequencing efficiency (i.e., mitochondrial and nuclear ribosomal RNAs and tRNAs). Probes for this RNA enrichment library were then synthesized (Twist Bioscience) for use with a standard target enrichment panel process.

Performance relative to uncaptured RNA-seq. Target capture was uniquely able to purify the subset of protein-coding genes. This design allowed for improved efficiency without the need for a ribosomal depletion step. The design outperformed whole transcriptome sequencing (WTS) and 3′ counting in having the least amount of intronic bases called and the most exonic content (expression profiling efficiency). More coding genes were detected with a lower 3′ bias and percent duplication rate (FIG. 40A). Coding sequences (CDS's) are generally the most informative part of a gene for detecting fusion events and are generally easier to uniquely assign reads to when compared with UTRs. As the RNA enrichment library is primarily designed against CDS's, substantially more coding reads (CDSs) were obtained relative to other techniques (FIG. 40B). Since capture uses a limited quantity of probes, a leveling effect was evaluated where capture probes could become saturated. However, comparing gene counts in a WTS sample to captured counts showed that enrichment is more or less even across the full 5 orders of magnitude of gene expression (FIG. 40C).

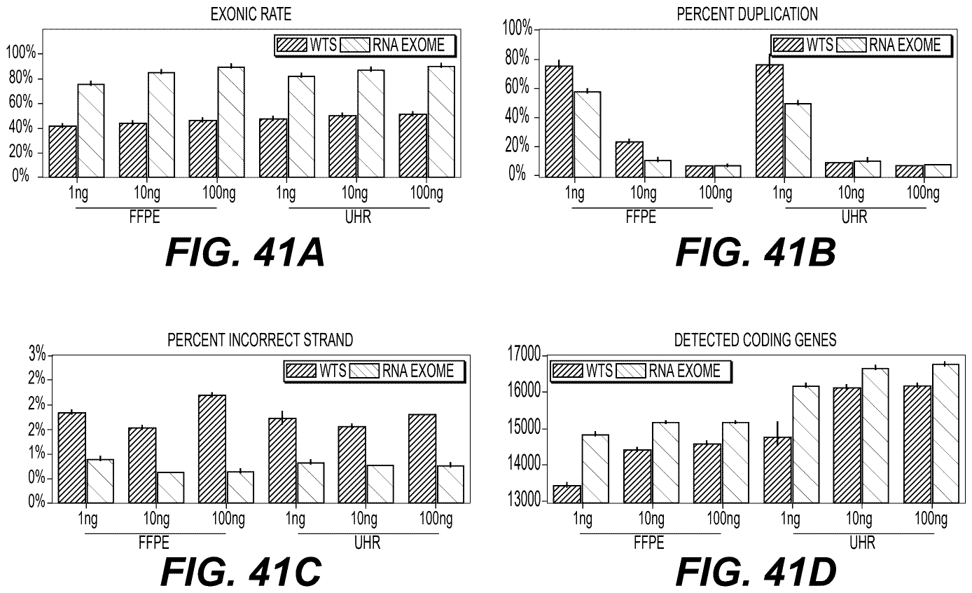

Capture of damaged/low-mass templates. Formalin-fixed paraffin-embedded (FFPE) tissue is tissue that has been preserved for histology. Although this process damages nucleic acids, FFPE tissue is nonetheless often used for RNA-seq because the samples are readily available as clinical specimens. FFPE tissues were then evaluated using the RNA enrichment library. Results indicated that the RNA exome enriches equally efficiently in FFPE as in non-FFPE samples (FIG. 41A), while reducing duplicate rates (FIG. 41B), reducing incorrect strand percent (FIG. 41C), and increasing the number of detected genes (FIG. 41D) compared to WTS.

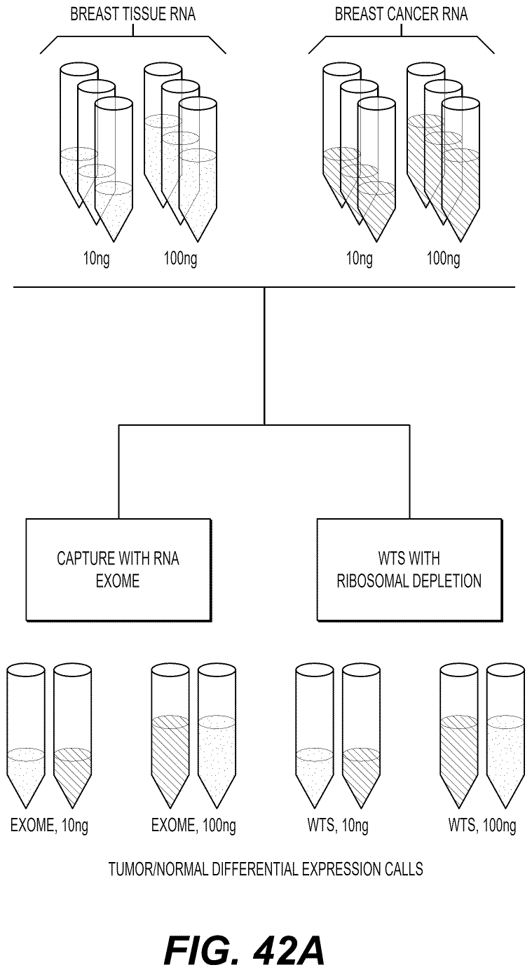

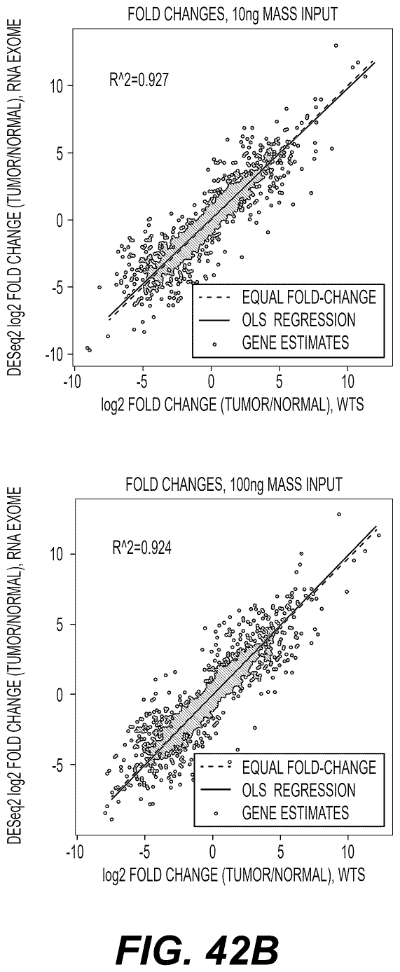

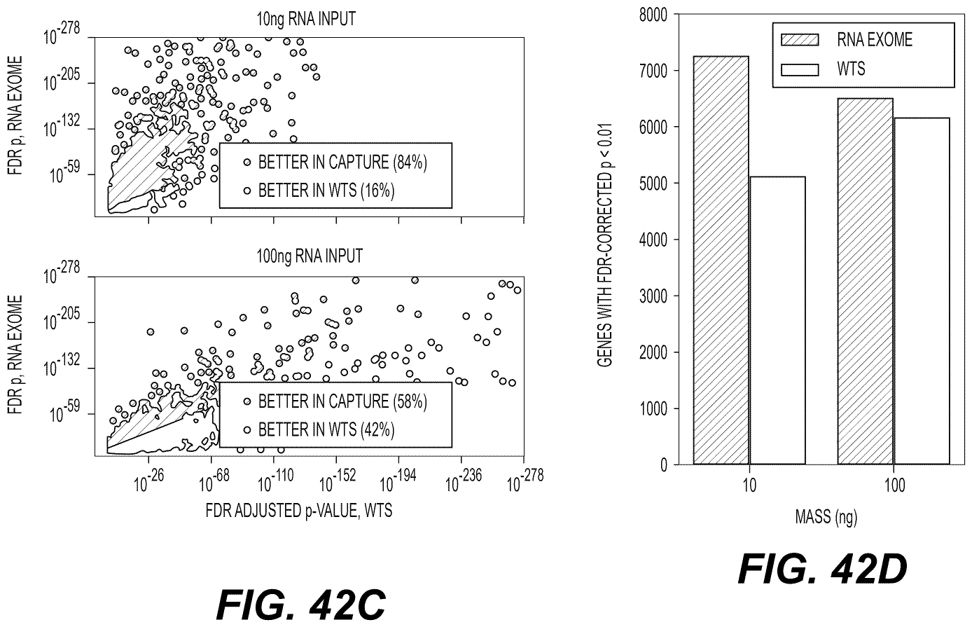

Differential expression. One important application of RNA sequencing, particularly in oncology applications, is differential expression. Although capture does introduce some bias into gene expression estimates (FIG. 40C), this bias was extremely consistent for the same genes between runs. Preservation of differences in gene expression for WTS and RNA exome capture were then evaluated. Three replicates of paired Tumor/Normal RNA reference samples were evaluated through both WTS and RNA exome capture (FIG. 42A), using both high-(100 ng) and low-input (10 ng) conditions to evaluate whether limited material behaves differently in capture and WTS. Differential expression estimates were similar between the two experimental workflows (FIG. 42B), but the increased read counts from capture provide better statistical power (FIG. 42C), and identified more genes that were significantly altered between the tumor and normal conditions (FIG. 42D).

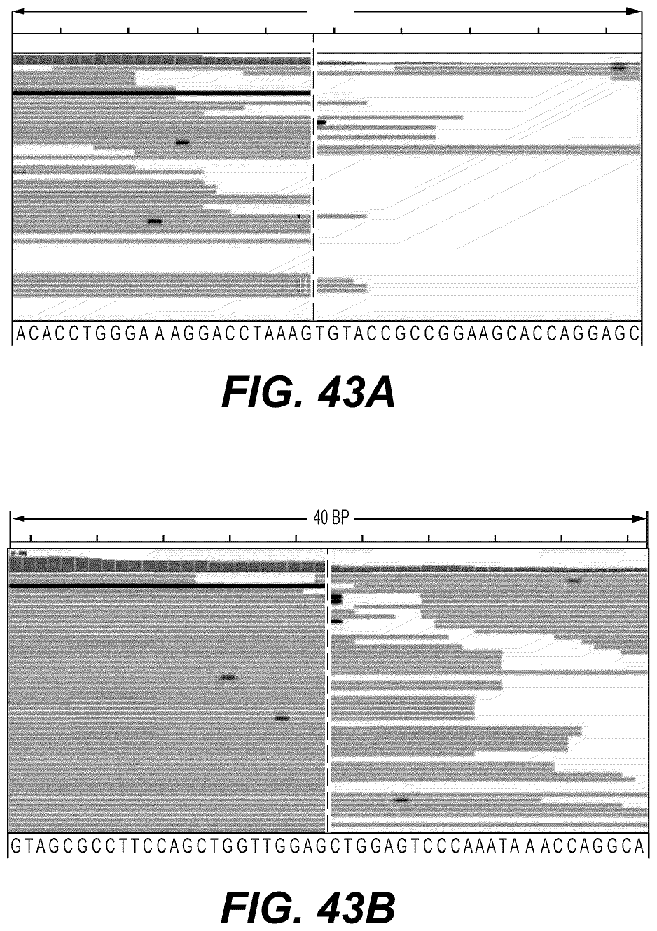

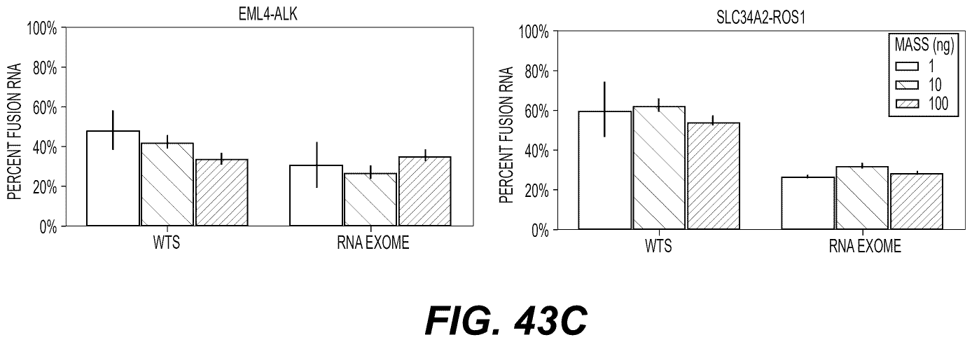

Fusion RNA detection. In addition to gene quantification, an important application of RNA-seq is to discover certain classes of structural variants (such as gene fusions) that are difficult to discover in DNA space. One potential challenge with RNA capture is that it might introduce bias towards transcripts in the design space and cause these fusion transcripts to be underrepresented. Material containing two fusions common in solid tumors (EML4-ALK and SLC34A2-ROS1) was sequenced and subjected to the RNA enrichment workflow. After mapping reads to the consensus sequences of the fusion variants, reads spanning the breakpoints (FIGS. 43A-43B) were evaluated. Fusion and normal transcripts were also quantified, and their ratios compared (FIG. 43C), showing that capture preserved detection of fusions across a range of mass conditions.

Materials and methods. To test the RNA enrichment library, Ing, 10 ng, or 100 ng of Universal Human Reference RNA (Agilent P/N 740000) or FFPE RNA Fusion Reference Standards (Horizon Discovery P/N HD784) was added to the RNA-seq Library Preparation Kit (Twist Bioscience). Prior to making libraries, FFPE material was extracted using the Qiagen RNeasy® FFPE Kit. Target enrichment was performed using 500 ng of library and the Target Enrichment Standard Hybridization v2 Protocol with a 16-hour hybridization reaction time. Sequencing was performed with the Illumina NextSeq platform and 76 bp paired-end reads. Analysis was performed by sampling FASTQ files to a fixed number of reads (10M pairs/20M reads unless otherwise specified). Alignment was performed against hg38 using STAR and gene quantification was performed using FeatureCounts with GenCode v41 gene annotations. Metrics were calculated using Picard CollectRnaSeqMetrics. Data processing and visualization were performed with Pandas and Seaborn using custom Python scripts. Genome browser visualization was performed with IGV. Fusion transcript quantification was performed using Salmon with an index built from the GenCode v41 transcript sequences concatenated to the fusion transcript sequences.

The present disclosure is further described by the following non-limiting items:

Item 1. A synthetic polynucleotide library comprising: a plurality of polynucleotides, wherein the polynucleotides comprise DNA and are configured to hybridize with one or more regions of target nucleic acids, and wherein the target nucleic acids comprise a cDNA library.

Item 2. The library of Item 1, wherein the cDNA library comprises at least one exon-exon boundary between a first exon and a second exon.

Item 3. The library of Item 1 or 2, wherein the plurality of polynucleotides comprises a first polynucleotide and a second polynucleotide, wherein the first and second polynucleotides do not span the at least one exon-exon boundary.

Item 4. The library of any one of Items 1-3, wherein at least one polynucleotide is configured to hybridize to the first exon, and at least one polynucleotide is configured to hybridize to the second exon.

Item 5. The library of any one of Items 1-4, wherein the plurality of polynucleotides comprise at least two polynucleotides which do not span at least 90% of exon-exon boundaries.

Item 6. The library of any one of Items 1-5, wherein the plurality of polynucleotides comprise at least two polynucleotides which do not span any exon-exon boundaries.

Item 7. The library of any one of Items 1-6, wherein the cDNA library is representative of at least 50,000 RNA transcripts.

Item 8. The library of any one of Items 1-6, wherein the cDNA library is representative of 25,000 to 100,000 RNA transcripts.

Item 9. The library of any one of Items 1-8, wherein the cDNA library is representative of at least 5,000 genes.

Item 10. The library of any one of Items 1-8, wherein the cDNA library is representative of at least 10,000 genes.

Item 11. The library of any one of Items 1-8, wherein the cDNA library is representative of 10,000 to 30,000 genes.

Item 12. The library of any one of Items 1-11, wherein the polynucleotides are 80-160 bases in length.

Item 13. The library of any one of Items 1-12, wherein the library comprises at least 50,000 polynucleotides.

Item 14. The library of any one of Items 1-13, wherein the library comprises at least 500,000 polynucleotides.

Item 15. The library of any one of Items 1-14, wherein the library comprises 100,000 to 750,000 polynucleotides.

Item 16. The library of any one of Items 1-15, wherein exon regions of the target nucleic acids encode for at least 500 genes.

Item 17. The library of Item 16, wherein a portion of the at least 500 genes comprises two or more isoforms.

Item 18. The library of any one of Items 1-17, wherein at least a portion of the polynucleotides is biotinylated.

Item 19. The library of any one of Items 1-18, wherein the library is configured to minimize hybridization with one or more housekeeping genes.

Item 20. The library of Item 19, wherein the one or more housekeeping genes comprise the highest 1.5% expressed genes in a cell.

Item 21. The library of any one of Items 1-20, wherein the target nucleic acids are derived from a human cell.

Item 22. The library of any one of Items 1-21, wherein the target nucleic acids are derived from an FFPE sample.

Item 23. The library of any one of Items 1-22, wherein the stoichiometry of the plurality of polynucleotides is adjusted based on mRNA transcript abundance.

Item 24. The library of any one of Items 1-23, wherein the polynucleotides are tiled over one or more exon regions.

Item 25. The library of any one of Items 1-24, wherein library hybridization bias is minimized towards one or more exon-exon junctions.

Item 26. A method for sequencing comprising: (a) contacting a library of any one of Items 1-25 with a sample comprising a plurality of target nucleic acids; (b) enriching at least one nucleic acid that binds to the library; and (c) sequencing the at least one enriched target nucleic acid.

Item 27. The method of Item 26, wherein the method further comprises generating the target nucleic acids from RNA.

Item 28. The method of Item 26 or 27, wherein the plurality of target nucleic acids comprise a cDNA library.

Item 29. The method of any one of Items 26-28, wherein the method does not comprise a ribosomal depletion step.

Item 30. The method of any one of Items 26-29, wherein sequencing results in no more than 10% intronic bases.

Item 31. The method of any one of Items 26-30, wherein sequencing results in no more than 2% rRNA bases.

Item 32. The method of any one of Items 26-31, wherein sequencing results in at least 80% expression profiling efficiency.

Item 33. The method of any one of Items 26-32, wherein sequencing results in no more 10% duplication.

Item 34. The method of any one of Items 26-33, wherein sequencing results in no more 1.5% incorrect read strands.

Item 35. The method of any one of Items 26-34, wherein sequencing results in no more 3% median 3′ bias.

Item 36. The method of any one of Items 26-35, wherein at least 40% of sequenced bases are coding DNA sequences (CDS).

Item 37. The method of any one of Items 26-36, wherein at least 40% of sequenced bases are coding DNA sequences (CDS).

Item 38. The method of any one of Items 26-37, wherein the plurality of target nucleic acids is no more than 100 ng.

Item 39. The method of any one of Items 26-37, wherein the plurality of target nucleic acids is no more than 10 ng.

Item 40. The method of any one of Items 26-39, wherein sequencing comprises detection of at least one RNA fusion.

While exemplary and representative embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A synthetic polynucleotide library comprising:
a plurality of polynucleotides, wherein the polynucleotides comprise DNA and are configured to hybridize with one or more regions of target nucleic acids, and wherein the target nucleic acids comprise a cDNA library.

2. The library of claim 1, wherein the cDNA library comprises at least one exon-exon boundary between a first exon and a second exon.

3. The library of claim 2, wherein the plurality of polynucleotides comprises a first polynucleotide and a second polynucleotide, wherein the first and second polynucleotides do not span the at least one exon-exon boundary.

4. The library of claim 3, wherein the first polynucleotide is configured to hybridize to the first exon, and the second polynucleotide is configured to hybridize to the second exon.

5. The library of claim 1, wherein the plurality of polynucleotides comprises at least two polynucleotides which do not span at least 90% of exon-exon boundaries.

6. The library of claim 1, wherein the plurality of polynucleotides comprises at least two polynucleotides which do not span any exon-exon boundaries.

7. The library of claim 1, wherein the cDNA library is representative of 25,000 to 100,000 RNA transcripts.

8. The library of claim 1, wherein the cDNA library is representative of at least 5,000 genes.

9. The library of claim 1, wherein the cDNA library is representative of 10,000 to 30,000 genes.

10. The library of claim 1, wherein each polynucleotide of the plurality of polynucleotides are 80-160 has a length of 80 bases to 160 bases in length.

11. The library of claim 1, wherein the library comprises at least 50,000 polynucleotides.

12. The library of claim 1, wherein the library comprises 100,000 to 750,000 polynucleotides.

13. The library of claim 1, wherein exon regions of the target nucleic acids encode for at least 500 genes.

14. The library of claim 13, wherein a portion of the at least 500 genes comprises two or more isoforms.

15. The library of claim 1, wherein the plurality of polynucleotides are configured to minimize hybridization with one or more housekeeping genes.

16. The library of claim 15, wherein the one or more housekeeping genes comprise a highest 1% of expressed genes in a cell.

17. The library of claim 1, wherein at least a portion of the plurality of polynucleotides are biotinylated.

18. The library of claim 1, wherein a stoichiometry of the plurality of polynucleotides corresponds to an mRNA transcript abundance.

19. The library of claim 4, wherein the plurality of polynucleotides further comprises a third polynucleotide configured to hybridize with the first exon and a fourth polynucleotide configured to hybridize with the second exon.

20. The library of claim 19, wherein a portion of the first polynucleotide configured to hybridize with the first exon is at a first position within the first polynucleotide, and a portion of the third polynucleotide configured to hybridize with the first exon is at a second position within the third polynucleotide, and the first position is different than the second position.
